(19) United States
(12) Patent Application Publication
(10) Pub. No.: US 2026/0347756 A1
(43) Pub. Date: Nov. 5, 2026

(54) ENGINEERED BACTERIAL STRAINS WITH MODIFIED SUGAR TRANSPORT FOR BIOPROCESSING APPLICATIONS

(71) Applicant: The Trustees of Princeton University, Princeton, NJ (US)

(72) Inventors: Jonathan Conway, Princeton, NJ (US); Hansen Tjo, Princeton, NJ (US)

(73) Assignee: The Trustees of Princeton University, Princeton, NJ (US)

(21) Appl. No.: 19/668,475

(22) Filed: May 5, 2026

Related U.S. Application Data
(60) Provisional application No. 63/799,978, filed on May 5, 2025.

(51) Int. Cl. C12N 1/20; C07K 14/195; C12N 1/16; C12N 9/14; C12R 1/01; C12R 1/645

(52) U.S. Cl. CPC C12N 1/20; C07K 14/195; C12N 1/16; C12N 9/14; C12Y 306/01003; C12R 2001/01; C12R 2001/645

(57) ABSTRACT

A genetically engineered bacterial strain comprising a genome comprising a dis of at least one sugar transporter gene locus, wherein the deletion reduces or eliminates the ability of the strain to utilize at least one sugar substrate for growth, and wherein the strain retains the ability to utilize at least one other sugar substrate for growth. Systems for bioprocessing are also provided, comprising a first genetically engineered bacterial strain and a second genetically engineered bacterial strain, each comprising deletions of different sugar transporter gene loci such that each strain selectively utilizes different sugar substrates for growth. Methods of controlling microbial growth in a bioprocessing application are further provided, comprising providing at least one genetically engineered bacterial strain with a sugar transporter gene locus deletion and growing the strain in a nutrient source containing sugar substrate(s), where growth is controlled based on the sugar substrates present.

### CROSS-REFERENCE TO RELATED APPLICATIONS

This application claims priority to U.S. Provisional Patent Application No. 63/799,978, titled “Engineered Sugar Utilization in Lignocellulolytic Thermopiles”, filed May 5, 2025, which is hereby incorporated by reference in its entirety.

### TECHNICAL FIELD

The present disclosure relates to genetically engineered bacterial strains with modified sugar transport systems for bioprocessing applications, and more particularly to thermophilic bacterial strains having disruptions or inactivations (including, e.g., deletions, insertions, frameshifts, etc.) of sugar transporter genes to enable selective sugar utilization in lignocellulosic biomass conversion and microbial co-culture systems.

### BACKGROUND

Biofuels and biochemicals derived from renewable feedstocks offer sustainable alternatives to petroleum-based products. Lignocellulosic biomass, which includes plant materials such as agricultural residues and dedicated energy crops, represents an abundant renewable resource for producing fuels and chemicals. However, the conversion of lignocellulosic biomass into useful products has been constrained by the physical and chemical recalcitrance of plant cell walls to degradation.

Thermophilic microorganisms, which thrive at elevated temperatures, have attracted interest for biomass conversion applications. These microbes operate at temperatures that may provide process advantages including enhanced reaction kinetics, reduced risks of bioreactor contamination, and lower energy costs for temperature control. Some thermophilic bacteria are capable of degrading the cellulose and hemicellulose components of lignocellulosic biomass into a diversity of oligosaccharide and monosaccharide sugars. Consolidated bioprocessing approaches, in which biomass degradation and sugar fermentation are combined in a single step, may eliminate the need for separate saccharification and fermentation process stages.

Sugar transport into microbial cells represents a step in the conversion of lignocellulosic biomass to metabolic products. Many bacteria and archaea utilize ATP-Binding Cassette (ABC) transporters to mediate sugar uptake. These transporters comprise a hydrophobic transmembrane protein domain, a cytoplasmic ATPase domain, and an extracellular substrate-binding domain that helps determine the sugar specificity of the associated transporter. While progress has been made in understanding ABC transporters in some model organisms, sugar transport remains relatively understudied in many non-model bacteria, particularly with respect to the diverse carbohydrates released from lignocellulose.

Microbial co-cultures have been deployed as a biotechnology strategy to increase chemical titers, rates, and yields by expanding metabolic capability. Synthetic microbial communities may unlock synergistic interactions that outperform monocultures. For example, different microbial strains may possess distinct enzyme inventories with functional complementarity in degrading cellulose and the diverse polysaccharides that make up hemicellulose. Co-culture strategies may also enable division of labor approaches, where one strain focuses on biomass deconstruction while another strain dedicates metabolic resources to product synthesis.

However, efforts to integrate thermophilic microorganisms as part of co-culture processes have faced challenges due to underdeveloped genetic engineering toolkits in these non-model microbes. The requirement of high temperature growth conditions has meant limited access to functional genetic parts and tools that are typically active under mesophilic conditions. Additionally, when multiple strains in a co-culture compete for the same sugar substrates, community stability and overall process efficiency may be compromised.

Recent advancements have produced new genetic parts and tools for modifying thermophilic microorganisms. These developments have expanded the possibilities for engineering strains with tailored sugar utilization profiles. By modifying sugar transport systems, it may be possible to create strains that selectively utilize particular sugars while leaving other sugars unconsumed. Such engineered strains could be deployed in co-culture systems where substrate specialization reduces competition between strains, or in processes where certain sugars are preserved for downstream utilization by other organisms.

Accordingly, there remains interest in developing improved approaches for engineering microbial sugar transport systems to enable selective sugar utilization across diverse bioprocessing applications and manufacturing conditions.

### SUMMARY

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

In one aspect, a genetically engineered bacterial strain is provided. The genetically engineered bacterial strain comprises a genome comprising a disruption or inactivation of at least one sugar transporter gene locus (such as a deletion), wherein the disruption or inactivation reduces or eliminates the ability of the strain to utilize at least one sugar substrate for growth, and wherein the strain retains the ability to utilize at least one other sugar substrate for growth. This configuration enables selective sugar utilization, which allows the engineered strain to be deployed in co-culture systems where substrate specialization reduces competition between strains, or in processes where certain sugars are preserved for downstream utilization by other organisms.

In some embodiments, the strain is a thermophilic bacterium. Thermophilic bacteria operate at elevated temperatures, yielding process advantages including enhanced reaction kinetics, reduced risks of bioreactor contamination, and lower energy costs for temperature control.

In some embodiments, the strain is derived from Anaerocellum bescii. Anaerocellum bescii possesses highly effective carbohydrate-active enzymes, an absence of carbon catabolite repression enabling simultaneous utilization of a broad range of substrates, and availability of a genetic system for metabolic engineering, making it especially promising for consolidated bioprocessing of lignocellulosic biomass.

In some embodiments, the at least one sugar transporter gene locus comprises a cello-oligosaccharide transporter locus, and the disruption or inactivation reduces or eliminates the ability of the strain to utilize cellobiose, cellodextrins, or cellulose for growth. This enables the creation of strains that can degrade lignocellulosic biomass while leaving cello-oligosaccharides unconsumed for subsequent utilization by other organisms in a bioprocessing system.

In some embodiments, the at least one sugar transporter gene locus comprises a monosaccharide transporter locus, and the disruption or inactivation reduces or eliminates the ability of the strain to utilize D-xylose or D-glucose for growth. This allows the engineered strain to be co-cultured with other strains that preferentially consume D-xylose or D-glucose, thereby eliminating growth source competition and improving community stability.

In some embodiments, the at least one sugar transporter gene locus comprises a monosaccharide transporter locus, and the disruption or inactivation reduces or eliminates the ability of the strain to utilize L-arabinose or D-galactose for growth. This enables substrate specialization where the engineered strain consumes orthogonal sugars relative to other strains in a co-culture, maximizing product yields and overall metabolic efficiency.

In some embodiments, the genome further comprises a disruption or inactivation of a gene encoding a promiscuous ATPase that powers a plurality of oligosaccharide ATP-Binding Cassette transporters, and the disruption or inactivation of the gene encoding the promiscuous ATPase in combination with the disruption or inactivation of the at least one sugar transporter gene locus eliminates the ability of the strain to utilize the at least one sugar substrate for growth. Combining the promiscuous ATPase disruption or inactivation with a sugar transporter disruption or inactivation provides complete elimination of growth on the targeted sugar substrates, which may be advantageous when partial growth impairment is insufficient for the intended bioprocessing application.

In some embodiments, the gene encoding the promiscuous ATPase is msmK. The msmK gene encodes an ATPase that powers multiple oligosaccharide transporters, and its disruption or inactivation in combination with monosaccharide transporter disruption or inactivation provides a mechanism to completely disrupt growth on selected monosaccharides.

In some embodiments, the at least one sugar transporter gene locus encodes one or more components of an ATP-Binding Cassette transporter. ATP-Binding Cassette transporters mediate sugar uptake in many bacteria and archaea, and targeting these transporters provides a mechanism for controlling sugar utilization specificity.

In some embodiments, the at least one sugar transporter gene locus encodes at least one substrate-binding protein and at least one transmembrane domain protein. The substrate-binding protein largely dictates an ABC transporter's substrate specificity by binding and delivering sugars in the extracellular space to the transmembrane entryway, and disruption or inactivation of genes encoding these components disrupts the sugar uptake pathway.

In another aspect, a system for bioprocessing is provided. The system comprises a first genetically engineered bacterial strain comprising a first disruption or inactivation of a first sugar transporter gene locus, wherein the first disruption or inactivation reduces or eliminates the ability of the first strain to utilize a first set of one or more sugar substrates for growth, and a second genetically engineered bacterial strain comprising a second disruption or inactivation of a second sugar transporter gene locus different from the first sugar transporter gene locus, wherein the second disruption or inactivation reduces or eliminates the ability of the second strain to utilize a second set of one or more sugar substrates for growth, the second set being different from the first set. This configuration enables co-cultures of strains that utilize orthogonal sugar substrates, where each strain can be specialized to consume particular sugars in a mixed feed and produce particular molecule outputs without competing for the same carbon sources.

In some embodiments, the first genetically engineered bacterial strain and the second genetically engineered bacterial strain are each derived from Anaerocellum bescii. Using strains derived from the same parent organism provides compatible growth temperatures and enables the creation of a mix-and-match set of strains that can be grown together with predictable behavior.

In some embodiments, the first disruption or inactivation reduces or eliminates the ability of the first strain to utilize D-xylose or D-glucose for growth, and the second disruption or inactivation reduces or eliminates the ability of the second strain to utilize L-arabinose or D-galactose for growth. This pairing enables co-cultures that feed on orthogonal sugars to avoid substrate competition, where varying the ratios of these sugars gives rise to distinct co-culture compositions.

In some embodiments, the system further comprises a nutrient source containing at least two different sugar substrates, wherein the first genetically engineered bacterial strain and the second genetically engineered bacterial strain each selectively utilizes a different sugar substrate from the nutrient source. This enables tailored substrate utilization that maximizes product yields and improves community stability by eliminating growth source competition.

In some embodiments, the nutrient source comprises a lignocellulosic biomass substrate. Lignocellulosic biomass provides a diverse range of sugars including pentose and hexose sugars in various oligomeric forms, and co-culture strains can collectively consume these substrates to maximize biomass conversion.

In some embodiments, the first genetically engineered bacterial strain, the second genetically engineered bacterial strain, or both, are configured to degrade the lignocellulosic biomass substrate to release one or more sugars. This enables consolidated bioprocessing whereby biomass degradation and sugar fermentation are combined, eliminating the need for separate saccharification and fermentation process stages.

In some embodiments, the first genetically engineered bacterial strain is engineered to produce a first target product and the second genetically engineered bacterial strain is engineered to produce a second target product different from the first target product. This division of labor approach reduces metabolic burden on each strain by allowing individual strains to specialize in synergistic, strain-specific functions that increase growth rates, product yields, and overall metabolic efficiency of the co-culture.

In some embodiments, a ratio of the first target product to the second target product is controlled by adjusting a ratio of sugar substrates provided to the first genetically engineered bacterial strain and the second genetically engineered bacterial strain. This provides a mechanism for controlling co-culture compositions and product ratios through sugar inputs, enabling flexible bioprocessing strategies.

In another aspect, a method of controlling microbial growth in a bioprocessing application is provided. The method comprises providing at least one genetically engineered bacterial strain comprising a disruption or inactivation (which may be, e.g., genomic encoded) of at least one sugar transporter gene locus, wherein the disruption or inactivation reduces or eliminates the ability of the strain to utilize at least one sugar substrate for growth while the strain retains the ability to utilize at least one other sugar substrate for growth, and growing the at least one genetically engineered bacterial strain in a nutrient source containing one or more sugar substrates, wherein growth of the at least one genetically engineered bacterial strain is controlled based on the one or more sugar substrates present in the nutrient source. As will be understood, “growing” here includes culturing if done under controlled defined conditions, but also includes growing under less controlled conditions as well. This approach enables growth control through sugar addition, providing a mechanism for regulating microbial populations in monoculture or mixed culture systems.

In some embodiments, the at least one genetically engineered bacterial strain is a thermophilic bacterium grown at a temperature of 55 degrees Celsius or higher. Growing at elevated temperatures provides process advantages including enhanced reaction kinetics, reduced risks of bioreactor contamination, and lower energy costs for temperature control.

In some embodiments, growing may include culturing a plurality of genetically engineered bacterial strains in the nutrient source, each strain of the plurality having a different sugar transporter gene locus disruption or inactivation, and a composition of the plurality of strains in the nutrient source is controlled by adjusting a ratio of sugar substrates in the nutrient source. This enables predictive control of co-culture compositions through sugar inputs, allowing for rational design of microbial consortia in which each strain exhibits a distinct sugar utilization profile and performs a dedicated metabolic function.

In some embodiments, the nutrient source comprises a lignocellulosic biomass substrate, and the at least one genetically engineered bacterial strain degrades the lignocellulosic biomass substrate to release one or more sugars. This enables strains that cannot utilize particular sugars to grow on and degrade plant lignocellulosic biomass while leaving behind particular sugars that can then be fed to another organism in a subsequent process stage.

In some embodiments, the at least one genetically engineered bacterial strain is derived from Anaerocellum bescii, and the at least one sugar transporter gene locus encodes one or more components of an ATP-Binding Cassette transporter. Anaerocellum bescii possesses multiple ABC sugar transporters with distinct sugar specificities, enabling the creation of strains with tailored sugar utilization profiles through targeted transporter disruption or inactivation.

In some embodiments, the genome of the at least one genetically engineered bacterial strain further comprises a disruption or inactivation of a gene encoding a promiscuous ATPase that powers a plurality of oligosaccharide ATP-Binding Cassette transporters, and the disruption or inactivation of the gene encoding the promiscuous ATPase in combination with the disruption or inactivation of the at least one sugar transporter gene locus eliminates the ability of the strain to utilize the at least one sugar substrate for growth. This combination of disruptions and/or inactivations provides complete elimination of growth on targeted sugar substrates, which may be advantageous when partial growth impairment from single transporter disruptions and/or inactivations is insufficient for the intended bioprocessing application.

In some embodiments, the at least one genetically engineered bacterial strain degrades the lignocellulosic biomass substrate while leaving unconsumed the at least one sugar substrate that the strain cannot utilize, and the unconsumed sugar substrate is available for utilization by a different organism in a subsequent process stage. This enables sequential bioprocessing strategies where a first organism deconstructs biomass and consumes certain sugars while preserving other sugars for a second organism that may possess superior capabilities for converting those preserved sugars into desired products.

In some embodiments, the different organism is a yeast. Yeast strains are well-established industrial organisms with robust fermentation capabilities and extensive genetic toolkits, and pairing engineered thermophilic bacteria that leave behind specific sugars with yeast that efficiently convert those sugars enables hybrid bioprocessing approaches that leverage the strengths of each organism.

The foregoing general description of the illustrative embodiments and the following detailed description thereof are merely exemplary aspects of the teachings of this disclosure and are not restrictive.

### BRIEF DESCRIPTION OF FIGURES

Non-limiting and non-exhaustive examples are described with reference to the following figures.

FIG. 1 is a schematic showing genomic organization of maltodextrin ATP-binding cassette (ABC) transporters and neighboring genes associated with amylose metabolism in Anaerocellum bescii.

FIG. 2 is a schematic showing genomic organization of cello-oligosaccharide ATP-binding cassette (ABC) transporters and neighboring genes associated with cellulose utilization in Anaerocellum bescii.

FIG. 3 is a graph showing interface energy deltas, corresponding to free energies of binding, for each simulated maltodextrin-binding protein and substrate combination. The rank-ordering of each protein-substrate combination is aligned with experimental results.

FIG. 4 is a graph showing interface energy deltas, as a proxy for free energies of binding, are shown for each simulated combination of cellooligosaccharide and CBP. The rank-order affinity of Athe_0597 and Athe_0598 with their respective cello-oligosaccharide substrates are aligned with experimental results.

FIGS. 5 and 6 are tables showing thermal stabilization, binding stoichiometry, and dissociation constants for the binding of Athe_0597 (FIG. 5) and Athe_0598 (FIG. 6) to three cellodextrins.

FIG. 7 is a schematic showing two CUT1 ABC transporters powered by the promiscuous MsmK ATPase facilitate uptake of aglucan substrates in A. bescii. The maltose/trehalose ABC transporter Athe_2308-2310 preferentially uptakes shorter maltodextrins up to maltotriose, while the maltodextrin ABC transporter (Athe_2574-2578) transporters longer-chained maltodextrins such as maltotetraose and maltopentaose.

FIG. 8 is a schematic showing A. bescii possesses twenty-one CUT1 oligosaccharide transporters that share a promiscuous ATPase, MsmK (Athe_1803). The two CUT2 monosaccharide transporters in A. bescii possess their own respective ATPases. Thus, disruption or inactivation of msmK in A. bescii would inactivate all oligosaccharide transporters but not the monosaccharide transporters.

FIG. 9 is a table showing engineered A. bescii strains with predicted monosaccharide and oligosaccharide uptake capabilities.

FIG. 10 is a set of graphs showing orthogonal sugar transport is feasible with engineered strains of A. bescii. Growth curves of MACB1018(ΔmsmK) , HTAB184(Δ2264-8) , and HTAB804(Δ2264-8ΔmsmK) on D-Xylose, D-Glucose, L-Arabinose, and D-Galactose. Growth of HTAB189(Δ0105-7) on D-Xylose and D-Glucose is also shown. Although HTAB184(Δ2264-8) displays slower growth on arabinose and galactose, the additional disruption or inactivation of msmK in HTAB804(Δ2264-8ΔmsmK) completely eliminates growth on arabinose and galactose.

FIG. 11 is a set of graphs showing growth curves of axenic, engineered A. bescii strains MACB1018, MACB1080, and HTAB187 on carbohydrate substrates glucose, maltose, cellobiose, and microcrystalline cellulose (Avicel). All growth curves were performed in triplicate. For growth curves on glucose, maltose, and cellobiose, each data point represents the mean Optical Density at 680 nm(OD₆₈₀) value. Error bars denote the standard deviation. Growth curves on Avicel were performed in triplicate, but time point samples are mixed prior to pellet protein quantitation as done previously.

FIG. 12 is a schematic showing biodesign strategies for synthetic, thermophilic communities. A) Carbon substrate specialization is one strategy basis for engineered thermophilic communities. Microbes can be genetically engineered for removal of specialized substrate uptake pathways, eliminating competition with other thermophiles in co-culture. Here, to avoid substrate competition, thermophilic microbes Strain A and Strain B grow on distinct sugars from lignocellulosic biomass. B) Synthetic thermophilic communities can leverage division of labor for more efficient consolidated bioprocessing of lignocellulosic biomass. “Degrader” strains are specialized for deconstruction of lignocellulosic biomass, while “Converter” strains, which are minimally involved in biomass degradation, instead focus on production of value-added chemicals. Splitting the roles of biomass degradation and conversion decreases the metabolic burden placed on each strain, improving bioprocessing efficiency. C) Complex biosynthetic pathways for bioproduction can be modularized across multiple strains. In the given example, a single strain is overburdened by multiple genetic manipulations i.e. one perturbation to its genome and expression of two heterologous enzymes (Enzyme I and Enzyme II) to generate the desired bioproduct. Burden sharing these genetic modifications across two strains, while ensuring any pathway intermediate can be transported into the strain containing the final production step, can result in higher product titers.

FIG. 13 is a schematic of a Cellodextrin Transporter Knockout Vector (Athe_0595-Athe_0598).

FIG. 14 is a schematic of a D-Xylose/D-Glucose Transporter Knockout Vector (Athe_0105-Athe_0107).

FIG. 15 is a schematic of a L-Arabinose/D-Galactose Transporter Knockout Vector (Athe_2264-Athe_2266).

FIG. 16 is a schematic of a MsmK Knockout Vector (pKHT026).

FIG. 17 is a schematic showing deletion of the Athe_0595-Athe_0598 Cellodextrin transporter.

FIG. 18 is an image confirming deletion of the Athe_0595-Athe_0598 Cellodextrin transporter.

FIG. 19 is a table showing biophysical characterization of the substrate-binding proteins associated with the two ABC CUT2 monosaccharide transporters in A. bescii. Athe_2264, as part of the Athe_2264-8 ABC transporter operon, binds tightly to L-Arabinose and D-Galactose, whereas Athe_0105, as part of the Athe_0105-7 ABC transporter operon, binds tightly to D-Xylose and D-Glucose.

FIG. 20 is a graph showing how Athe_0105-Athe_0107 deletion causes D-Xylose/D-Glucose growth disruption.

FIG. 21 is a graph showing how Athe_2264-Athe_2266 deletion causes L-Arabinose/D-Galactose growth disruption.

FIG. 22 is a set of graphs showing genetic elimination of sugar uptake in engineered A. bescii strains.

FIG. 23 is a schematic showing the Phosphotransferase System (PTS) for fructose transport in A. bescii.

FIG. 24 is a graph showing growth curves confirming the PTS plays a role in fructose uptake.

FIG. 25 is a table showing a library of engineered A. bescii strains with their respective growth phenotypes on seven different sugar substrates including fructose, glucose, xylose, arabinose, galactose, maltose, and cellobiose.

FIG. 26 shows a co-culture growth curve demonstrating that a mixture of strains HTAB 804 and HTAB 809 results in a growing culture when inoculated at a 50/50 ratio.

FIG. 27 shows qPCR tracking of strain fraction over time.

FIG. 28 shows HPLC quantification of sugar content in the co-culture medium over time.

FIG. 29 is a growth curve for strain HTAB149.

### DETAILED DESCRIPTION

The following description sets forth exemplary aspects of the present disclosure. It should be recognized, however, that such description is not intended as a limitation on the scope of the present disclosure. Rather, the description also encompasses combinations and modifications to those exemplary aspects described herein.

The present disclosure relates to genetically engineered bacterial strains with modified sugar transport systems for bioprocessing applications. A genetically engineered bacterial strain may comprise a genome having a disruption or inactivation of at least one sugar transporter gene locus, wherein the disruption or inactivation reduces or eliminates the ability of the strain to utilize at least one sugar substrate for growth while the strain retains the ability to utilize at least one other sugar substrate for growth. By tailoring sugar uptake specificity through targeted disruptions and/or inactivations of sugar transporter genes, the engineered strains may be deployed in co-culture systems where substrate specialization reduces competition between strains, or in processes where certain sugars are preserved for downstream utilization by other organisms.

As used herein, ‘disruption’ and ‘inactivation’ refer, interchangeably, to any genetic, transcriptional, translational, or post-translational modification that eliminates, prevents, or substantially reduces the expression of a gene or the activity of its encoded product. Disruption or inactivation includes, without limitation: (i) complete or partial deletion of the gene, including deletion of coding sequence, regulatory sequence, or both; (ii) insertion of one or more nucleotides or heterologous sequence into the gene or its regulatory elements; (iii) substitution mutations, including missense, nonsense, and frameshift mutations; (iv) modification of promoter, enhancer, splice site, or other regulatory elements such that gene expression is abolished or substantially reduced; (v) replacement of the endogenous gene with a non-functional or reduced-function variant; (vi) conditional or inducible loss-of-function systems, including site-specific recombination; (vii) post-transcriptional silencing by RNA interference, antisense, or related mechanisms; (viii) CRISPR-mediated knockout, CRISPR interference, or base editing producing a non-functional product; and (ix) epigenetic silencing. A gene is considered disrupted or inactivated where the expression level or functional activity of the encoded product is reduced by at least 50%, at least 75%, at least 90%, or eliminated, relative to a corresponding unmodified gene. In some preferred embodiments, the gene is deleted.

In some aspects, the genetically engineered bacterial strain is a thermophilic bacterium. Thermophilic bacteria operate at elevated temperatures, providing process advantages including enhanced reaction kinetics, reduced risks of bioreactor contamination, and lower energy costs for temperature control. In some cases, the genetically engineered bacterial strain is derived from Anaerocellum bescii, an extremely thermophilic, lignocellulose-degrading bacterium. Anaerocellum bescii possesses highly effective carbohydrate-active enzymes (CAZymes), an absence of carbon catabolite repression enabling simultaneous utilization of a broad range of substrates, and availability of a genetic system for metabolic engineering. These attributes make A. bescii suitable for consolidated bioprocessing of lignocellulosic biomass, whereby biomass degradation and sugar fermentation are combined in a single step. In some aspects, the parent strain for genetic modifications is Anaerocellum bescii MACB1018. In some cases, the genetically engineered bacterial strain is a strain that is preferably grown at a growth temperature of 75 to 78 degrees Celsius.

In preferred aspects, the thermophilic bacterium may be selected from an Anaerocellum species or a Caldicellulosiruptor species. In some aspects, the thermophilic bacterium may be selected from the thermophilic bacterium may be selected from: Anaerocellum bescii, Caldicellulosiruptor bescii, Caldicellulosiruptor saccharolyticus, Caldicellulosiruptor kronotskyensis, Caldicellulosiruptor obsidiansis, or combinations thereof.

In some aspects, other species may be utilized. For example, the species may include a Clostridium species (e.g., Clostridium thermocellum), a Thermoanaerobacterium species (e.g., Thermoanaerobacterium saccharolyticum), a Thermoanaerobacter species (e.g., Thermoanaerobacter ethanolicus), and/or a Thermotoga species (e.g., Thermotoga maritima). These thermophilic bacteria are capable of growth at elevated temperatures and possess carbohydrate-active enzymes suitable for lignocellulosic biomass conversion.

Anaerocellum bescii possesses multiple ATP-Binding Cassette (ABC) sugar transporters that mediate uptake of diverse carbohydrates released from lignocellulose. ABC transporters comprise a hydrophobic transmembrane protein domain, a cytoplasmic ATPase domain, and an extracellular substrate-binding domain that helps determine the sugar specificity of the associated transporter. The ABC sugar transporters may be classified into two families based on structural and functional characteristics. Carbohydrate-Uptake-Transporter Family 1 (CUT1) transporters include a heterodimeric transmembrane pore made of two different proteins and preferentially uptake oligosaccharides. Carbohydrate-Uptake-Transporter Family 2 (CUT2) transporters utilize a homodimeric transmembrane barrel and preferentially uptake monosaccharides. For both transporter types, hydrolysis of two ATP molecules by the intracellular ATPase powers the translocation of one sugar molecule across the cell membrane.

By engineering the sugar transporter inventory in, a thermophilic bacterial strain, such as A. bescii, through targeted gene disruptions and/or inactivations, strains may be created that selectively transport and utilize particular sugars while leaving other sugars unconsumed. Such engineered strains may be mixed and matched to convert complex biomass into targeted end products, enabling a flexible strategy for high-temperature biorefining. The approach supports the development of customizable microbial communities in which each strain exhibits a distinct sugar utilization profile and performs a dedicated metabolic function. Co-cultures of strains that utilize orthogonal sugar substrates may each be specialized to consume particular sugars in a mixed feed and produce particular molecule outputs from the sugars the strains can consume. In some cases, strains that cannot utilize particular sugars may grow on and degrade plant lignocellulosic biomass while leaving behind particular sugars that may then be fed to another organism in a subsequent process stage.

A genetically engineered bacterial strain may comprise a genome comprising a disruption or inactivation of at least one sugar transporter gene locus. The disruption or inactivation reduces or eliminates the ability of the strain to utilize at least one sugar substrate for growth. The strain retains the ability to utilize at least one other sugar substrate for growth. By tailoring sugar uptake specificity through targeted disruption or inactivation of sugar transporter genes, the engineered strains may be deployed in co-culture systems where substrate specialization reduces competition between strains, or in processes where certain sugars are preserved for downstream utilization by other organisms.

In some aspects, the at least one sugar transporter gene locus encodes one or more components of an ATP-Binding Cassette transporter. ATP-Binding Cassette transporters mediate sugar uptake in many bacteria and archaea. ATP-Binding Cassette transporters comprise a hydrophobic transmembrane protein domain, a cytoplasmic ATPase domain, and an extracellular substrate-binding domain that helps determine the sugar specificity of the associated transporter.

In some aspects, the at least one sugar transporter gene locus encodes at least one substrate-binding protein and at least one transmembrane domain protein. The substrate-binding protein is responsible for binding and delivering sugars in the extracellular space to the transmembrane entryway, and the substrate-binding protein largely dictates an ABC transporter's substrate specificity. Substrate-binding proteins may contain two domains connected by a hinge region, where the presence of an appropriate ligand drives flexion about the hinge region and movement of one domain towards the other to capture the ligand in a binding pocket. With a bound substrate, the substrate-binding protein may adopt a closed and more thermally stable conformation. The transmembrane domain proteins form a heterodimeric transmembrane pore in Carbohydrate-Uptake-Transporter Family 1 (CUT1) transporters or a homodimeric transmembrane barrel in Carbohydrate-Uptake-Transporter Family 2 (CUT2) transporters, through which sugar molecules are translocated across the cell membrane.

Knockout strains may be constructed using homologous recombination. In some aspects, the method of constructing knockout strains uses a first-crossover step via kanamycin selection and a second-crossover step via 5-fluoro-orotic acid counter-selection. During the first-crossover step, a knockout vector integrates into the chromosome adjacent to the target gene locus. Single colonies may be selected and screened via PCR to confirm knockout vector chromosomal integration. A PCR-verified first crossover colony may then be plated on nutrient source containing 5-fluoro-orotic acid for counter-selection. Single colonies may be picked and screened for complete disruption or inactivation of the target locus following counter-selection via colony PCR.

In some aspects, knockout vectors contain approximately 1 kb flanking regions upstream and downstream of the target gene locus for homologous recombination. The flanking regions may be PCR amplified from genomic DNA and inserted into a plasmid backbone. Both flanking regions may be positioned adjacent to one another in the knockout vector. The upstream flanking region may be positioned upstream of the first gene in the target locus, and the downstream flanking region may be positioned downstream of the last gene in the target locus. Following homologous recombination and counter-selection, the intervening genes between the flanking regions are deleted from the chromosome, resulting in a strain that lacks the target sugar transporter gene locus.

In some aspects, the genetically engineered bacterial strain is a thermophilic bacterium. Thermophilic bacteria operate at elevated temperatures, providing process advantages including enhanced reaction kinetics, reduced risks of bioreactor contamination, and lower energy costs for temperature control.

In some aspects, the genetically engineered bacterial strain is derived from Anaerocellum bescii. Anaerocellum bescii is an extremely thermophilic, lignocellulose-degrading bacterium with an optimum growth temperature of approximately 75 to 78 degrees Celsius. A. bescii possesses highly effective carbohydrate-active enzymes (CAZymes) that efficiently degrade cellulose and hemicellulose into oligosaccharides. A. bescii exhibits an absence of carbon catabolite repression, enabling simultaneous utilization of a broad range of substrates including pentose and hexose sugars. A. bescii also has availability of a genetic system for metabolic engineering. These attributes make A. bescii suitable for consolidated bioprocessing of lignocellulosic biomass, whereby biomass degradation and sugar fermentation are combined in a single step. A. bescii may be genetically engineered to produce chemical commodities such as acetone, ethanol, and acetoin from lignocellulose.

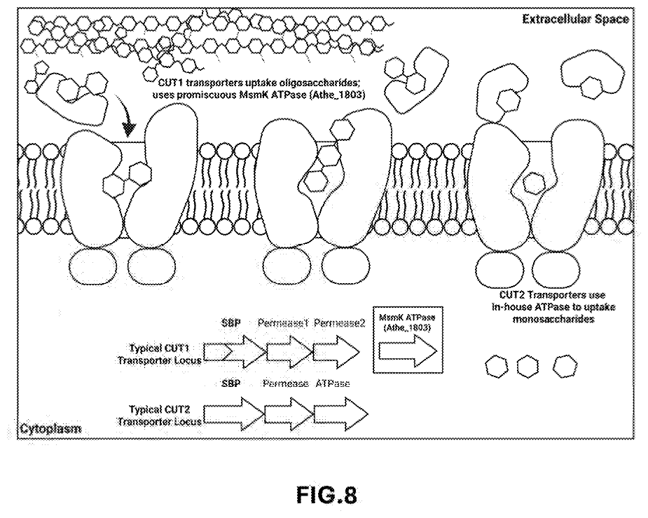

FIG. 8 illustrates the ATP-Binding Cassette (ABC) sugar transporter inventory in A. bescii. As shown in FIG. 8, A. bescii possesses twenty-one Carbohydrate-Uptake-Transporter Family 1 (CUT1) oligosaccharide transporters that share a promiscuous ATPase, MsmK, encoded by gene locus Athe_1803. The CUT1 transporters include a heterodimeric transmembrane pore made of two different proteins and preferentially uptake oligosaccharides. As further shown in FIG. 8, A. bescii possesses two Carbohydrate-Uptake-Transporter Family 2 (CUT2) monosaccharide transporters that possess their own respective ATPases. The CUT2 transporters utilize a homodimeric transmembrane barrel and preferentially uptake monosaccharides. Disruption or inactivation of msmK in A. bescii inactivates all oligosaccharide transporters but does not inactivate the monosaccharide transporters, as the CUT2 transporters possess their own ATPases. This architecture provides a mechanism for selectively disrupting oligosaccharide transport while preserving monosaccharide transport capabilities, or for combining msmK disruption or inactivation with monosaccharide transporter disruption or inactivation to achieve complete elimination of growth on targeted monosaccharides.

In some aspects, the at least one sugar transporter gene locus may comprise a cello-oligosaccharide transporter locus, and the disruption or inactivation may reduce or eliminate the ability of the strain to utilize cellobiose, cellodextrins, or cellulose for growth. As described above, A. bescii possesses multiple ATP-Binding Cassette (ABC) sugar transporters that mediate uptake of diverse carbohydrates released from lignocellulose. Among these transporters, a cello-oligosaccharide transporter locus encodes components for uptake of cello-oligosaccharides released from cellulose degradation.

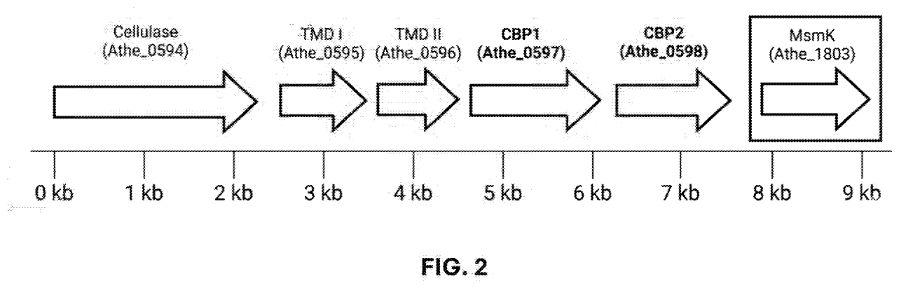

FIG. 2 illustrates the genomic organization of a cello-oligosaccharide ABC transporter locus and neighboring genes associated with cellulose utilization in A. bescii. As shown in FIG. 2, the cello-oligosaccharide transporter locus Athe_0595-0598 comprises genes encoding two transmembrane domain proteins (Athe_0595 and Athe_0596) and two substrate-binding proteins (Athe_0597 and Athe_0598). The transmembrane domain proteins Athe_0595 and Athe_0596 form a heterodimeric transmembrane pore through which cello-oligosaccharide molecules are translocated across the cell membrane. The substrate-binding proteins Athe_0597 and Athe_0598 are extracellular cello-oligosaccharide-binding proteins that bind and deliver cello-oligosaccharides to the transmembrane entryway. As further shown in FIG. 2, the cello-oligosaccharide transporter locus is co-localized with Athe_0594, which encodes an extracellular cellulase that degrades cellulose into glucose and cello-oligosaccharide products. The cello-oligosaccharide transporter locus is powered by the promiscuous MsmK ATPase encoded by Athe_1803, which is located at a different genomic position.

The cello-oligosaccharide transporter locus Athe_0595-0598 is conserved across the Anaerocellum and Caldicellulosiruptor genera. The substrate-binding protein Athe_0597 is highly conserved across both genera, with the C. morganii homolog possessing the highest sequence deviation from the A. bescii version while still retaining an amino acid sequence identity of greater than 94.8%. The substrate-binding protein Athe_0598 exhibits higher sequence variability, particularly among Caldicellulosiruptor species.

In some aspects, the genetically engineered bacterial strain is Anaerocellum bescii strain HTAB187 comprising a disruption or inactivation of the Athe_0595-0598 locus. Strain HTAB187 comprises a disruption or inactivation of the cello-oligosaccharide transporter locus including the substrate-binding proteins Athe_0597 and Athe_0598 and the transmembrane domain proteins Athe_0595 and Athe_0596.

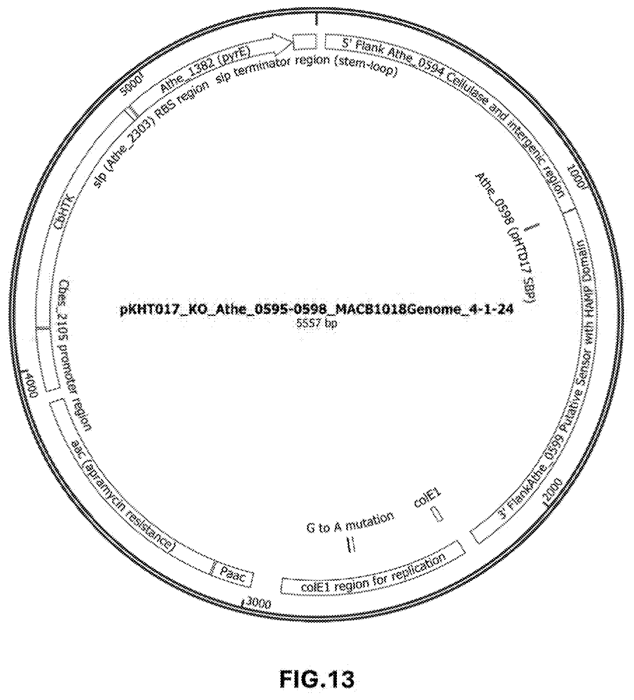

FIG. 13 illustrates a knockout vector for deleting the cello-oligosaccharide transporter locus Athe_0595-0598. As shown in FIG. 13, the knockout vector pKHT017 contains a flanking region upstream of the Athe_0595 gene and a flanking region downstream of the Athe_0598 gene. Both flanking regions are positioned adjacent to one another in the knockout vector. Following homologous recombination and counter-selection, the intervening genes Athe_0595, Athe_0596, Athe_0597, and Athe_0598 are deleted from the chromosome, resulting in strain HTAB187 that lacks the cello-oligosaccharide transporter locus.

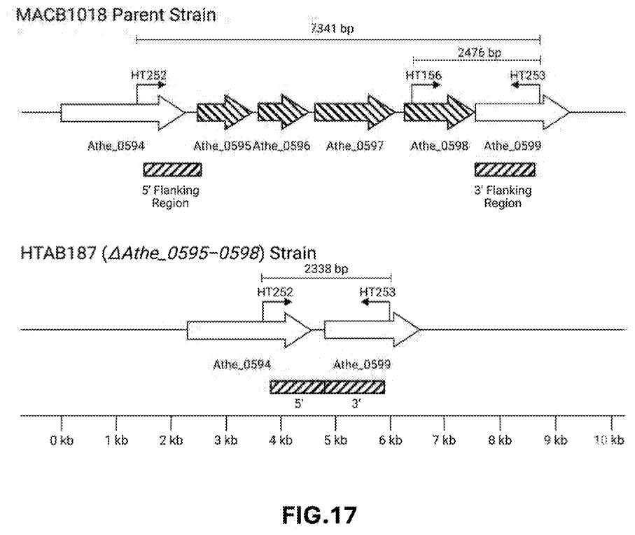

FIG. 17 illustrates the genomic organization of the parent strain MACB1018 and the deletion strain HTAB187. As shown in FIG. 17, the parent strain MACB1018 contains the complete cello-oligosaccharide transporter locus Athe_0595-0598 positioned between the flanking genes Athe_0594 and Athe_0599. In strain HTAB187, the Athe_0595-0598 locus has been deleted, leaving only the flanking genes Athe_0594 and Athe_0599 with the flanking regions joined at the deletion junction.

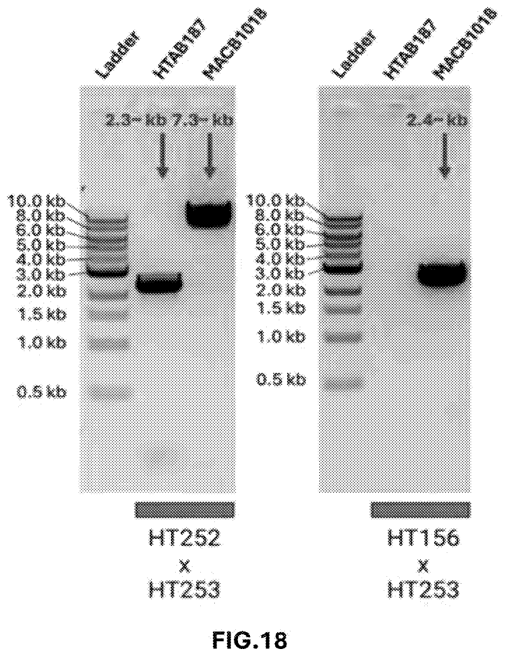

FIG. 18 illustrates PCR verification confirming deletion of the Athe_0595-0598 cello-oligosaccharide transporter locus in strain HTAB187. As shown in FIG. 18, PCR amplification across the deletion junction produces a smaller amplicon in strain HTAB187 compared to the parent strain MACB1018, confirming successful deletion of the transporter locus.

Deletion of the Athe_0595-0598 cello-oligosaccharide transporter locus in strain HTAB187 results in impairment of growth on cellobiose and elimination of growth on cellulose. Strain HTAB187 exhibits reduced growth on cellobiose compared to the parent strain MACB1018, though the growth defect does not prevent growth entirely on cellobiose. Strain HTAB187 is incapable of growth on cellulose. Strain HTAB187 retains the ability to grow on other carbohydrate substrates such as glucose and maltose, demonstrating that the deletion of the cello-oligosaccharide transporter locus does not impair uptake of other glucose-based substrates. This configuration enables the creation of strains that may degrade lignocellulosic biomass while leaving cello-oligosaccharides unconsumed for subsequent utilization by other organisms in a bioprocessing system.

In some aspects, the approaches described herein for engineering sugar transport systems in A. bescii may be applied to other thermophilic bacteria. The strategy of deleting sugar transporter gene loci to create strains with selective sugar utilization profiles may be extended to other organisms that possess ABC sugar transporters with defined substrate specificities. For bacteria that utilize CUT1 and CUT2 transporter families, identification of substrate-binding proteins and their cognate sugars through biophysical characterization enables rational selection of transporter loci for disruption or inactivation. In organisms that possess a promiscuous ATPase powering multiple oligosaccharide transporters, disruption or inactivation of the ATPase gene may be combined with monosaccharide transporter disruption or inactivation to achieve complete elimination of growth on targeted sugars. The genetic engineering approach using homologous recombination with flanking regions and counter-selection may be adapted to other thermophiles for which genetic systems are available, including Clostridium thermocellum, Thermoanaerobacterium saccharolyticum, Caldicellulosiruptor saccharolyticus, and other members of the Caldicellulosiruptor genus. The conservation of the cello-oligosaccharide transporter locus across, e.g., the Anaerocellum and Caldicellulosiruptor genera suggests that similar disruptions or inactivations may produce analogous phenotypes in related species.

As used herein, the term “related species” refers to bacterial species that share phylogenetic ancestry and exhibit conserved genomic features, including homologous gene loci encoding functionally similar proteins. Related species may belong to the same genus or to closely related genera within the same taxonomic family. For example, species within the Caldicellulosiruptor genus are related species that share conserved sugar transporter loci with Anaerocellum bescii.

In some aspects, a bacterial species qualifies as a related species when it exhibits one or more of the following features: sequence identity of at least 70% for 16S ribosomal RNA genes compared to a reference species; conservation of syntenic gene clusters encoding sugar transport systems; presence of homologous substrate-binding proteins with amino acid sequence identity of at least 50%; shared utilization of similar carbohydrate substrates; and comparable optimal growth temperature ranges. For thermophilic bacteria, related species may additionally share the architectural feature of a promiscuous ATPase powering multiple oligosaccharide transporters, enabling similar genetic engineering strategies to be applied across species.

The cello-oligosaccharide transporter locus Athe_0595-0598 includes two substrate-binding proteins, Athe_0597 and Athe_0598, that bind and deliver cello-oligosaccharides to the transmembrane entryway. Biophysical characterization of these substrate-binding proteins reveals distinct substrate specificities and binding thermodynamics.

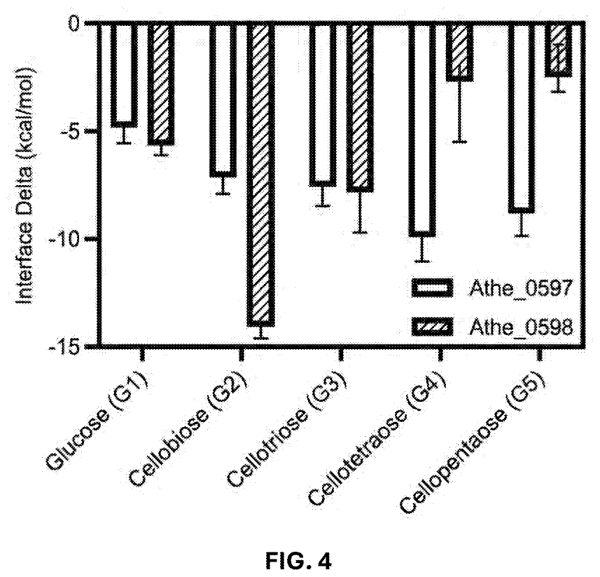

FIG. 4 illustrates interface energy deltas, as a proxy for free energies of binding, for each simulated combination of cello-oligosaccharide and substrate-binding protein. As shown in FIG. 4, the substrate-binding protein Athe_0597 exhibits favorable binding energies with cello-oligosaccharides of varying lengths from cellobiose (G2) to cellopentaose (G5). The substrate-binding protein Athe_0598 exhibits favorable binding energy with cellobiose (G2) but does not exhibit favorable binding with larger cello-oligosaccharides.

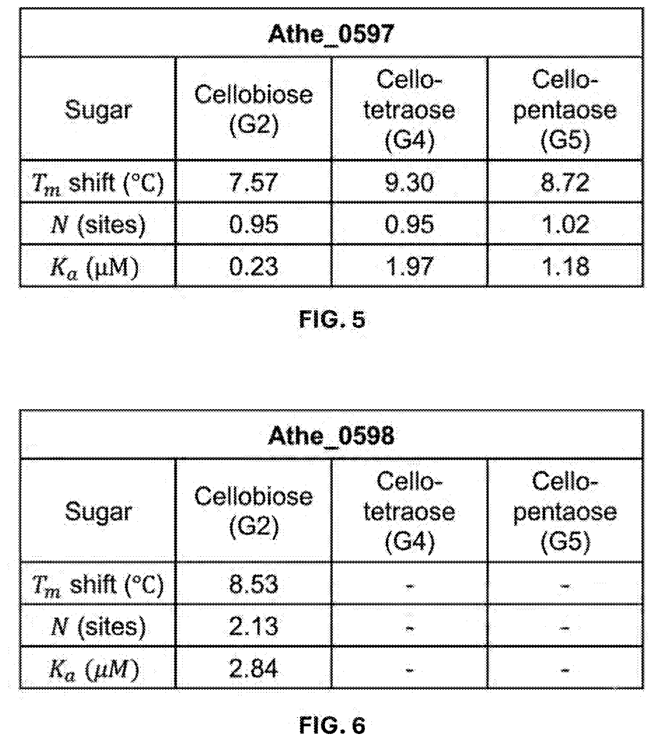

FIG. 5 illustrates thermal stabilization, binding stoichiometry, and dissociation constants for the binding of Athe_0597 to cellodextrins. As shown in FIG. 5, the substrate-binding protein Athe_0597 binds cellobiose (G2) with a dissociation constant of 0.23 micromolar, cellotetraose (G4) with a dissociation constant of 1.97 micromolar, and cellopentaose (G5) with a dissociation constant of 1.18 micromolar. The binding stoichiometry values are approximately 1.0 for all tested cello-oligosaccharides, indicating a 1:1 binding ratio between the substrate-binding protein and the cello-oligosaccharide ligand. The melting temperature shifts range from 7.57 degrees Celsius for cellobiose to 9.30 degrees Celsius for cellotetraose, indicating thermal stabilization upon ligand binding.

FIG. 6 illustrates thermal stabilization, binding stoichiometry, and dissociation constants for the binding of Athe_0598 to cellodextrins. As shown in FIG. 6, the substrate-binding protein Athe_0598 binds cellobiose (G2) with a dissociation constant of 2.84 micromolar and a melting temperature shift of 8.53 degrees Celsius. The substrate-binding protein Athe_0598 does not exhibit detectable binding to cellotetraose (G4) or cellopentaose (G5), as indicated by the absence of measurable binding parameters for these larger cello-oligosaccharides. The substrate-binding protein Athe_0598 is specific to cellobiose and does not bind larger cello-oligosaccharides.

The binding of cello-oligosaccharides to substrate-binding proteins Athe_0597 and Athe_0598 is entropically driven. In isothermal titration calorimetry measurements, TΔS values exceed ΔH values in all measured substrate-binding protein and cello-oligosaccharide combinations. The enthalpies of binding for Athe_0597 with cello-oligosaccharides are endothermic. The entropic contributions to binding may arise from the release of water molecules from the protein binding site upon ligand binding, where hydrophobic amino acids in the binding pocket subsites become desolvated and the release of water to the bulk solvent leads to a net increase in solvent translational entropy.

The substrate-binding proteins adopt a closed conformation when bound to cognate substrates through a Venus Fly-trap mechanism. In the absence of a bound ligand, the substrate-binding proteins exist in an open, apo state with a native melting temperature. When bound to cognate substrates, the substrate-binding proteins acquire a closed conformation with higher thermal stability. The presence of the appropriate ligand drives flexion about a hinge region connecting two domains of the substrate-binding protein, causing movement of one domain towards the other to capture the ligand in the binding pocket. The difference in melting temperatures between the apo and holo states indicates the sugar specificity of a given substrate-binding protein, where sugars with higher affinity yield greater melting temperature shifts.

In some aspects, the at least one sugar transporter gene locus may comprise a monosaccharide transporter locus, and the disruption or inactivation may reduce or eliminate the ability of the strain to utilize D-xylose or D-glucose for growth. As described above, A. bescii possesses two Carbohydrate-Uptake-Transporter Family 2 (CUT2) monosaccharide transporters that mediate uptake of monosaccharides. The CUT2 transporters utilize a homodimeric transmembrane barrel and preferentially uptake monosaccharides. Unlike the CUT1 oligosaccharide transporters that share the promiscuous MsmK ATPase, the CUT2 monosaccharide transporters possess their own respective ATPases.

The monosaccharide transporter locus Athe_0105-0107 encodes components for uptake of D-xylose and D-glucose. The locus includes a substrate-binding protein Athe_0105 and transmembrane domain proteins that form the transporter complex. The substrate-binding protein Athe_0105 binds and delivers D-xylose and D-glucose in the extracellular space to the transmembrane entryway, and the substrate-binding protein largely dictates the transporter's substrate specificity.

Biophysical characterization of the substrate-binding protein Athe_0105 demonstrates tight binding to D-xylose and D-glucose. The substrate-binding protein Athe_0105 binds D-xylose with a dissociation constant of approximately 0.10 micromolar and binds D-glucose with a dissociation constant of approximately 0.11 micromolar. The melting temperature shift for Athe_0105 with D-xylose is 9.57 degrees Celsius, and the melting temperature shift with D-glucose is 9.0 degrees Celsius. The binding stoichiometry values are approximately 0.76 for D-xylose and 0.93 for D-glucose, indicating approximately 1:1 binding ratios between the substrate-binding protein and the monosaccharide ligands. The dissociation constants in the sub-micromolar range indicate high affinity binding characteristic of ABC sugar transporter systems.

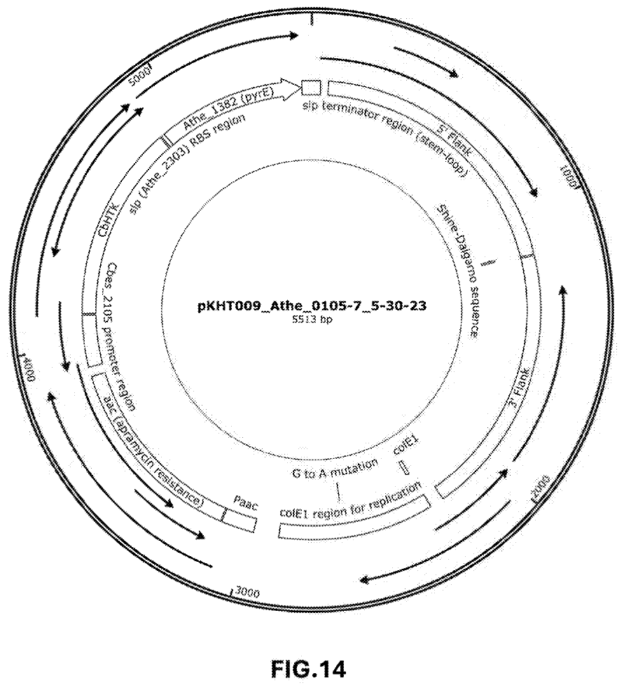

FIG. 14 illustrates a knockout vector for deleting the monosaccharide transporter locus Athe_0105-0107. As shown in FIG. 14, the knockout vector pKHT009 contains a flanking region upstream of the Athe_0105 gene and a flanking region downstream of the Athe_0107 gene. Both flanking regions are positioned adjacent to one another in the knockout vector. Following homologous recombination and counter-selection, the intervening genes Athe_0105, Athe_0106, and Athe_0107 are deleted from the chromosome.

In some aspects, the genetically engineered bacterial strain is Anaerocellum bescii strain HTAB189 comprising a deletion of the Athe_0105-0107 locus. Strain HTAB189 comprises a deletion of the monosaccharide transporter locus including the substrate-binding protein Athe_0105 and the transmembrane domain proteins Athe_0106 and Athe_0107.

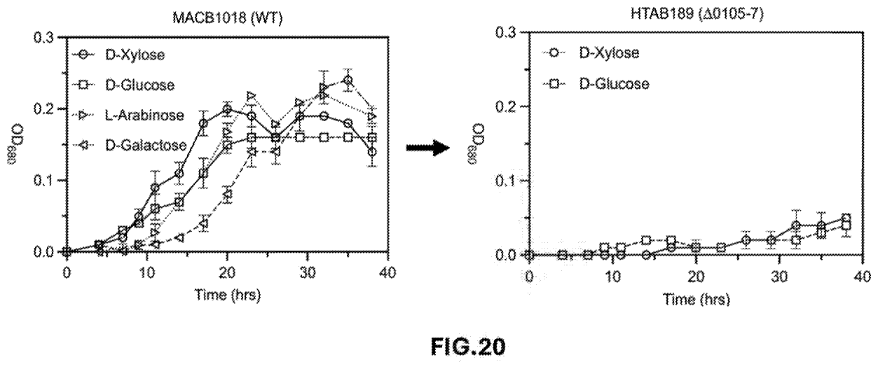

FIG. 20 illustrates growth curves comparing the wild-type strain MACB1018 and the deletion strain HTAB189 on monosaccharide substrates. As shown in FIG. 20, the wild-type strain MACB1018 exhibits growth on D-xylose, D-glucose, L-arabinose, and D-galactose. In contrast, strain HTAB189 carrying the Athe_0105-0107 deletion exhibits disrupted growth on D-xylose and D-glucose compared to the parent strain MACB1018. The deletion of the Athe_0105-0107 monosaccharide transporter locus in strain HTAB189 results in impairment of growth on D-xylose and D-glucose. This configuration enables the creation of strains that may be co-cultured with other strains that preferentially consume D-xylose or D-glucose, thereby reducing growth source competition and improving community stability in bioprocessing applications.

In some aspects, the at least one sugar transporter gene locus may comprise a monosaccharide transporter locus, and the disruption or inactivation may reduce or eliminate the ability of the strain to utilize L-arabinose or D-galactose for growth. As described above, A. bescii possesses two Carbohydrate-Uptake-Transporter Family 2 (CUT2) monosaccharide transporters that mediate uptake of monosaccharides. The CUT2 transporters utilize a homodimeric transmembrane barrel and preferentially uptake monosaccharides. Unlike the CUT1 oligosaccharide transporters that share the promiscuous MsmK ATPase, the CUT2 monosaccharide transporters possess their own respective ATPases.

The monosaccharide transporter locus Athe_2264-2268 encodes components for uptake of L-arabinose and D-galactose. The locus includes a substrate-binding protein Athe_2264 and transmembrane domain proteins that form the transporter complex. The substrate-binding protein Athe_2264 binds and delivers L-arabinose and D-galactose in the extracellular space to the transmembrane entryway, and the substrate-binding protein largely dictates the transporter's substrate specificity.

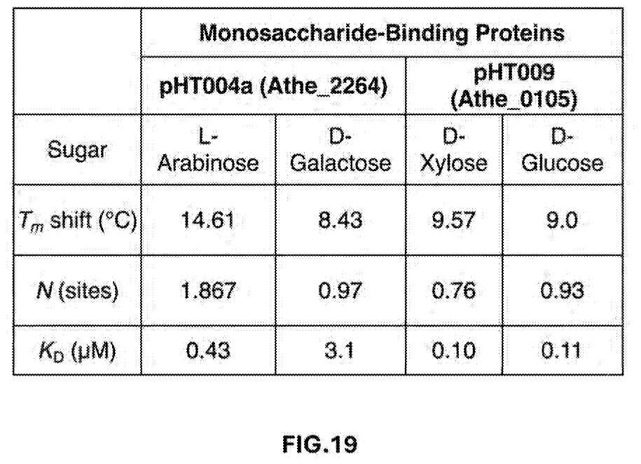

FIG. 19 illustrates biophysical characterization of the substrate-binding proteins associated with the two ABC CUT2 monosaccharide transporters in A. bescii. As shown in FIG. 19, the substrate-binding protein Athe_2264, as part of the Athe_2264-2268 ABC transporter operon, binds L-arabinose and D-galactose. The substrate-binding protein Athe_2264 binds L-arabinose with a dissociation constant of approximately 0.43 micromolar and binds D-galactose with a dissociation constant of approximately 3.1 micromolar. The melting temperature shift for Athe_2264 with L-arabinose is 14.61 degrees Celsius, and the melting temperature shift with D-galactose is 8.43 degrees Celsius. The binding stoichiometry values are approximately 1.867 for L-arabinose and 0.97 for D-galactose, indicating approximately 1:1 binding ratios between the substrate-binding protein and the monosaccharide ligands. The dissociation constants in the sub-micromolar to low micromolar range indicate high affinity binding characteristic of ABC sugar transporter systems.

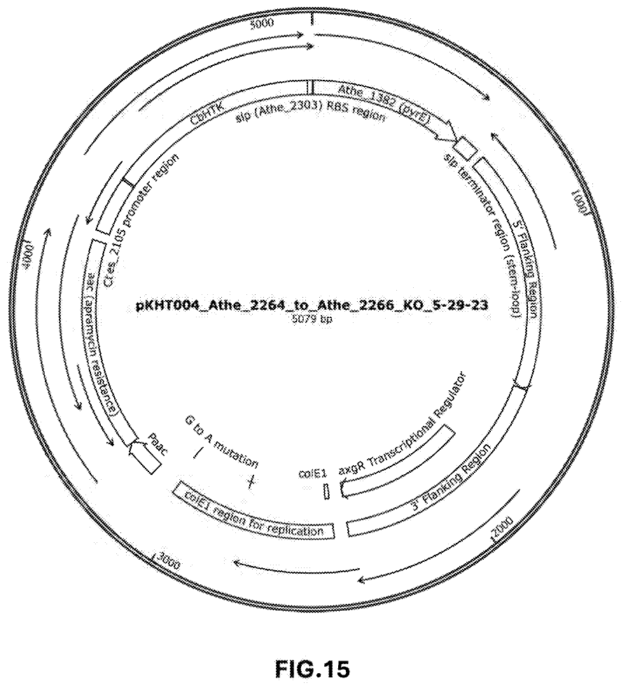

FIG. 15 illustrates a knockout vector for deleting the monosaccharide transporter locus Athe_2264-2266. As shown in FIG. 15, the knockout vector pKHT004 contains a flanking region upstream of the Athe_2264 gene and a flanking region downstream of the Athe_2266 gene. Both flanking regions are positioned adjacent to one another in the knockout vector. Following homologous recombination and counter-selection, the intervening genes Athe_2264, Athe_2265, and Athe_2266 are deleted from the chromosome.

In some aspects, the genetically engineered bacterial strain is Anaerocellum bescii strain HTAB184 comprising a deletion of the Athe_2264-2268 locus. Strain HTAB184 comprises a deletion of the monosaccharide transporter locus including the substrate-binding protein Athe_2264 and the transmembrane domain proteins.

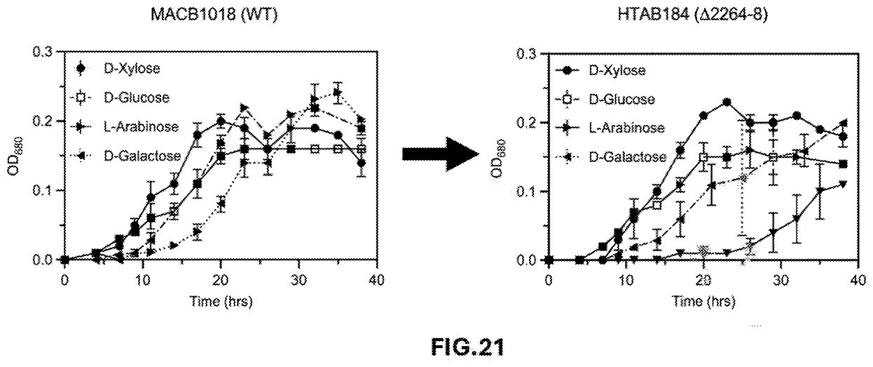

FIG. 21 illustrates growth curves comparing the wild-type strain MACB1018 and the deletion strain HTAB184 on monosaccharide substrates. As shown in FIG. 21, the wild-type strain MACB1018 exhibits growth on D-xylose, D-glucose, L-arabinose, and D-galactose. In contrast, strain HTAB184 carrying the Athe_2264-2268 deletion exhibits disrupted growth on L-arabinose and D-galactose compared to the parent strain MACB1018. Strain HTAB184 displays slower growth on L-arabinose and D-galactose while maintaining growth on D-xylose and D-glucose. The deletion of the Athe_2264-2268 monosaccharide transporter locus in strain HTAB184 results in impairment of growth on L-arabinose and D-galactose. This configuration enables the creation of strains that may be co-cultured with other strains that preferentially consume L-arabinose or D-galactose, thereby reducing growth source competition and improving community stability in bioprocessing applications.

As described above, the genome of a genetically engineered bacterial strain may further comprise a disruption or inactivation of a gene encoding a promiscuous ATPase that powers a plurality of oligosaccharide ATP-Binding Cassette transporters. The disruption or inactivation of the gene encoding the promiscuous ATPase in combination with the disruption or inactivation of the at least one sugar transporter gene locus may eliminate the ability of the strain to utilize the at least one sugar substrate for growth. In some aspects, the gene encoding the promiscuous ATPase is msmK.

In A. bescii, the promiscuous ATPase MsmK encoded by Athe_1803 powers twenty-one Carbohydrate-Uptake-Transporter Family 1 (CUT1) oligosaccharide transporters. Deletion of msmK (Athe_1803) has been shown to eliminate growth on oligosaccharides such as cellobiose. However, deletion of msmK (Athe_1803) does not prevent growth on monosaccharides, as the Carbohydrate-Uptake-Transporter Family 2 (CUT2) monosaccharide transporters possess their own respective ATPases. Among the twenty-one oligosaccharide transporters powered by MsmK, some may have slight affinity for monosaccharides. This affinity may explain why single transporter deletions of Athe_0105-0107 or Athe_2264-2268 fail to completely eliminate growth on the monosaccharides that the deleted transporters have been determined to uptake. Combining a monosaccharide transporter disruption or inactivation with disruption or inactivation of msmK (Athe_1803) may completely eliminate growth on the selected monosaccharides.

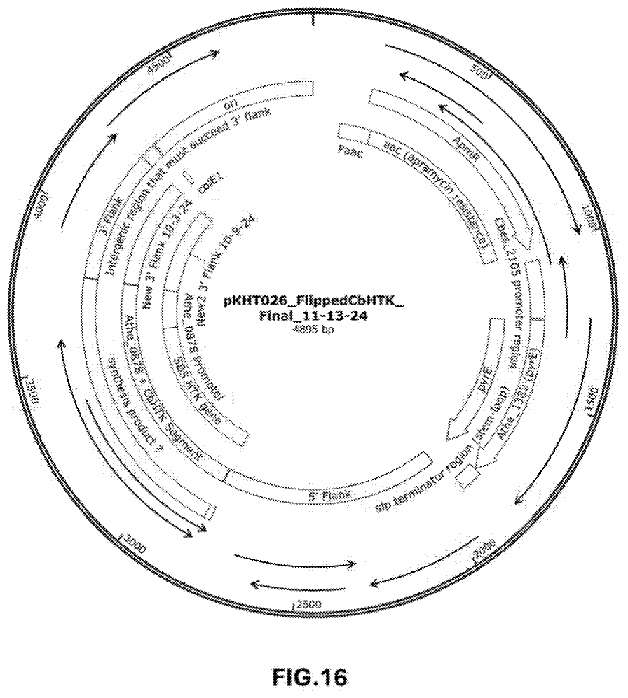

FIG. 16 illustrates a knockout vector for deleting the msmK gene (Athe_1803). As shown in FIG. 16, the knockout vector pKHT026 contains flanking regions for homologous recombination to delete the msmK gene from the chromosome. Following homologous recombination and counter-selection, the msmK gene is deleted from the chromosome, resulting in inactivation of all oligosaccharide transporters that depend on the MsmK ATPase for function.

In some aspects, the genetically engineered bacterial strain is Anaerocellum bescii strain HTAB804 comprising deletions of both the Athe_2264-2268 locus and msmK. Strain HTAB804 comprises a deletion of the monosaccharide transporter locus Athe_2264-2268 in combination with a deletion of the msmK gene encoding the promiscuous ATPase. Strain HTAB804 does not grow on L-arabinose and D-galactose. The combination of the Athe_2264-2268 deletion with the msmK deletion eliminates the ability of the strain to utilize L-arabinose and D-galactose for growth.

In some aspects, the genetically engineered bacterial strain is Anaerocellum bescii strain HTAB809 comprising deletions of both the Athe_0105-0107 locus and msmK. Strain HTAB809 comprises a deletion of the monosaccharide transporter locus Athe_0105-0107 in combination with a deletion of the msmK gene encoding the promiscuous ATPase. Strain HTAB809 does not grow on D-xylose and D-glucose. The combination of the Athe_0105-0107 deletion with the msmK deletion eliminates the ability of the strain to utilize D-xylose and D-glucose for growth.

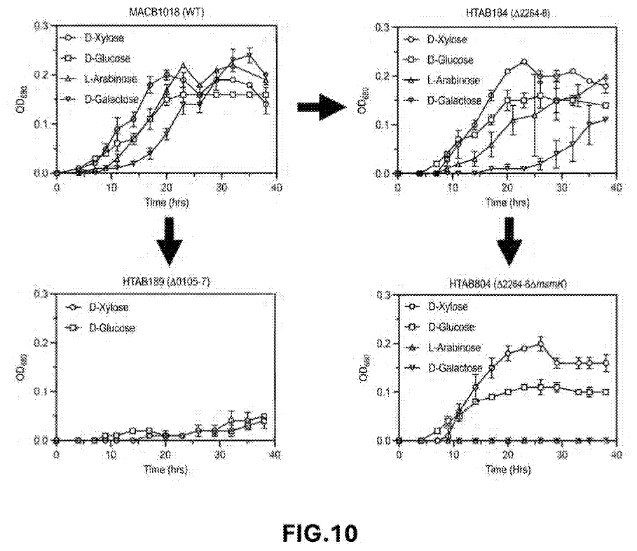

FIG. 10 illustrates growth curves demonstrating orthogonal sugar transport with engineered strains of A. bescii. As shown in FIG. 10, growth curves of strain HTAB184 carrying the Athe_2264-2268 deletion and strain HTAB804 carrying both the Athe_2264-2268 deletion and the msmK deletion are compared on D-xylose, D-glucose, L-arabinose, and D-galactose. Growth of strain HTAB189 carrying the Athe_0105-0107 deletion on D-xylose and D-glucose is also shown. Although strain HTAB184 displays slower growth on L-arabinose and D-galactose compared to the wild-type strain, the additional deletion of msmK in strain HTAB804 completely eliminates growth on L-arabinose and D-galactose. Strain HTAB804 retains the ability to grow on D-xylose and D-glucose, demonstrating that the combination of the Athe_2264-2268 deletion with the msmK deletion selectively eliminates growth on L-arabinose and D-galactose while preserving growth on other monosaccharides.

The combination of a monosaccharide transporter disruption or inactivation with the msmK disruption or inactivation provides a mechanism for achieving complete elimination of growth on targeted monosaccharides. This approach may be advantageous when partial growth impairment from single transporter disruption or inactivation is insufficient for the intended bioprocessing application. Strains with complete elimination of growth on specific monosaccharides may be deployed in co-culture systems where substrate specialization reduces competition between strains, or in processes where certain sugars are preserved for downstream utilization by other organisms.

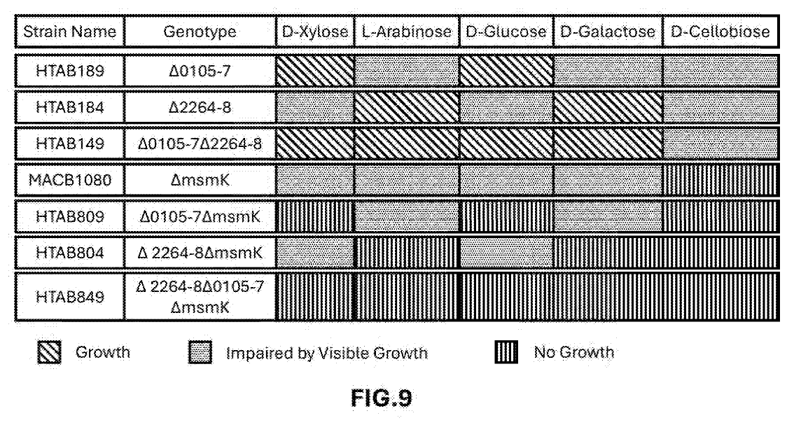

FIG. 9 illustrates engineered A. bescii strains with predicted monosaccharide and oligosaccharide uptake capabilities. As shown in FIG. 9, combinatorial disruption or inactivation of the two monosaccharide transporters result in various defects to growth on four different monosaccharides, as well as oligosaccharides including cellobiose.

In some aspects, the genetically engineered bacterial strain is Anaerocellum bescii strain HTAB149 comprising disruption or inactivation of both the Athe_0105-0107 locus and the Athe_2264-2268 locus. Strain HTAB149 comprises disruption or inactivation of both monosaccharide transporter loci, including the substrate-binding protein Athe_0105 that binds D-xylose and D-glucose, and the substrate-binding protein Athe_2264 that binds L-arabinose and D-galactose. Strain HTAB149 retains an active msmK gene encoding the promiscuous ATPase. Strain HTAB149 exhibits impaired growth on D-xylose, D-glucose, L-arabinose, and D-galactose. The dual monosaccharide transporter deletions in strain HTAB149 result in growth defects on all four monosaccharides, though growth may still be possible due to low-affinity transport through the remaining Carbohydrate-Uptake-Transporter Family 1 (CUT1) oligosaccharide transporters powered by MsmK.

In some aspects, the genetically engineered bacterial strain is Anaerocellum bescii strain HTAB849 comprising deletions of the Athe_0105-0107 locus, the Athe_2264-2268 locus, and msmK. Strain HTAB849 comprises deletions of both monosaccharide transporter loci in combination with a deletion of the msmK gene encoding the promiscuous ATPase that powers twenty-one CUT1 oligosaccharide transporters. Strain HTAB849 does not grow on D-xylose, D-glucose, L-arabinose, or D-galactose. The combination of the dual monosaccharide transporter deletions with the msmK deletion eliminates the ability of the strain to utilize D-xylose, D-glucose, L-arabinose, and D-galactose for growth. Strain HTAB849 is defective for uptake of all monosaccharides transported by the ABC transporter systems.

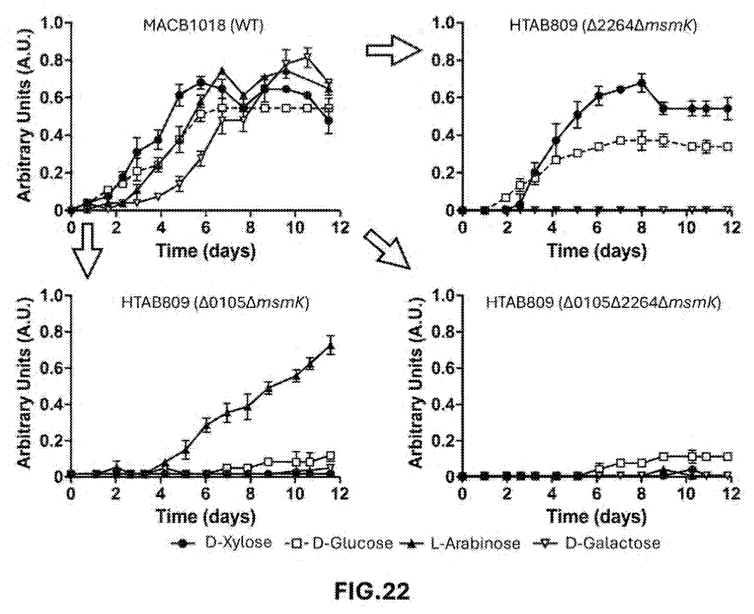

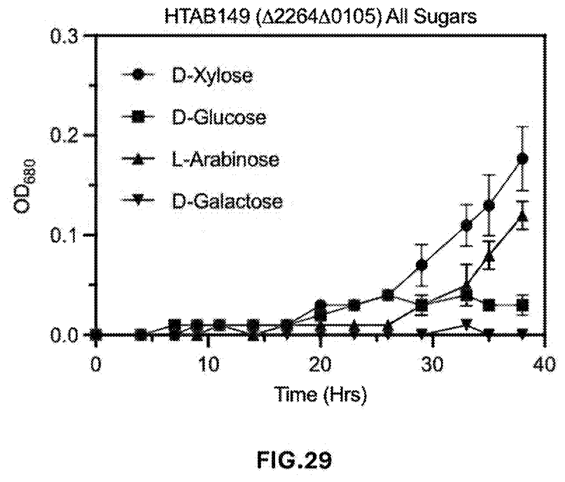

In some aspects, the genetically engineered bacterial strain retains the ability to grow on fructose via a Phosphotransferase System (PTS) transporter even when sugar transporter gene loci are deleted. A. bescii possesses twenty-three ABC sugar transporters and a twenty-fourth sugar transporter that is a Phosphotransferase System (PTS) transporter for fructose transport. The PTS transporter operates independently of the ABC transporter systems and is not affected by disruption or inactivation of the ABC sugar transporter gene loci or the msmK gene. Strains with disruption or inactivation of the monosaccharide transporter loci Athe_0105-0107 and Athe_2264-2268, including strains HTAB149 and HTAB849, may retain the ability to grow on fructose as long as the PTS locus remains intact (see, e.g., FIGS. 22, 25, 29). This configuration enables the creation of strains that are defective for uptake of D-xylose, D-glucose, L-arabinose, D-galactose, and oligosaccharides, while retaining the ability to utilize fructose as a carbon source.

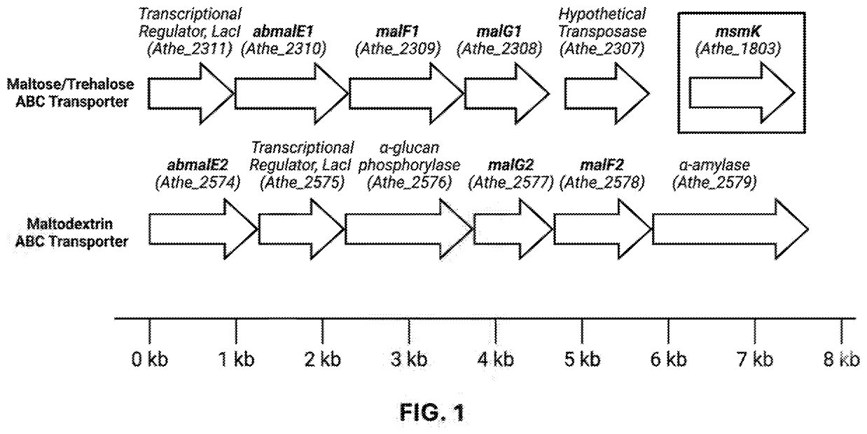

A. bescii possesses two orthogonal ATP-Binding Cassette (ABC) transporters that mediate maltodextrin uptake. FIG. 1 illustrates the genomic organization of maltodextrin ABC transporters and neighboring genes associated with amylose metabolism in A. bescii. As shown in FIG. 1, a maltose/trehalose ABC transporter locus includes a transcriptional regulator (Athe_2311), a substrate-binding protein gene (Athe_2310), and two transmembrane domain genes (Athe_2309 and Athe_2308). A maltodextrin ABC transporter locus includes a substrate-binding protein gene (Athe_2574), a transcriptional regulator (Athe_2575), an alpha-glucan phosphorylase gene (Athe_2576), two transmembrane domain genes (Athe_2577 and Athe_2578), and an alpha-amylase gene (Athe_2579). The maltodextrin ABC transporter locus contains neighboring genes associated with alpha-glucan metabolism, indicating a role in maltodextrin uptake.

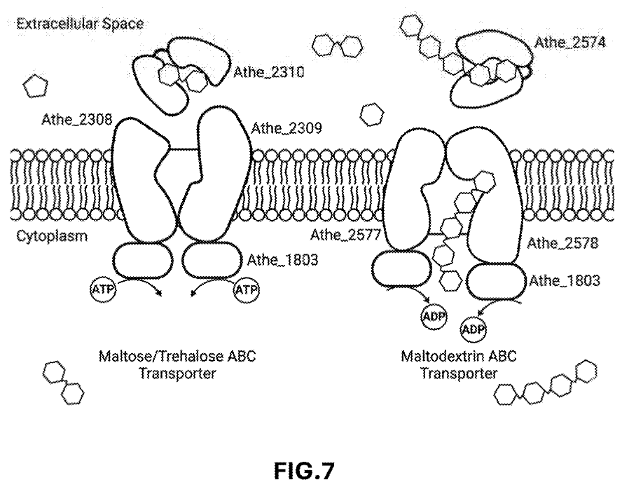

FIG. 7 illustrates two Carbohydrate-Uptake-Transporter Family 1 (CUT1) ABC transporters powered by the promiscuous MsmK ATPase that facilitate uptake of alpha-glucan substrates in A. bescii. As shown in FIG. 7, the maltose/trehalose ABC transporter comprising Athe_2308-2310 preferentially uptakes shorter maltodextrins up to maltotriose, while the maltodextrin ABC transporter comprising Athe_2574-2578 transports longer-chained maltodextrins such as maltotetraose and maltopentaose. Both ABC transporters are powered by the promiscuous MsmK ATPase encoded by Athe_1803.

The substrate-binding proteins Athe_2310 and Athe_2574 exhibit distinct substrate specificities that enable orthogonal maltodextrin uptake. The substrate-binding protein Athe_2310may bind maltose and trehalose with dissociation constants in the micromolar range, preferring shorter maltodextrins. Biophysical characterization using differential scanning calorimetry demonstrates that Athe_2310 exhibits the largest melting temperature shifts for maltose and trehalose, with progressively smaller thermal stabilization for longer maltodextrins. Isothermal titration calorimetry measurements indicate that Athe_2310 binds trehalose, maltose, and maltotriose with micromolar dissociation constants. Athe_2310 does not exhibit binding to longer maltodextrins such as maltotetraose, maltopentaose, or maltoheptaose.

The substrate-binding protein Athe_2574 may bind longer maltodextrins from maltotetraose to maltoheptaose with dissociation constants in the sub-micromolar range. Differential scanning calorimetry demonstrates that Athe_2574 exhibits the largest melting temperature shifts with longer oligosaccharides including maltotetraose, maltopentaose, and maltoheptaose. Isothermal titration calorimetry measurements indicate that Athe_2574 preferentially binds longer malto-oligosaccharides, with the highest affinity for maltoheptaose. The shift in binding affinity from maltotriose to maltotetraose is marked, with dissociation constants an order of magnitude lower observed for Athe_2574 and long malto-oligosaccharides compared to Athe_2310 and short maltodextrins.

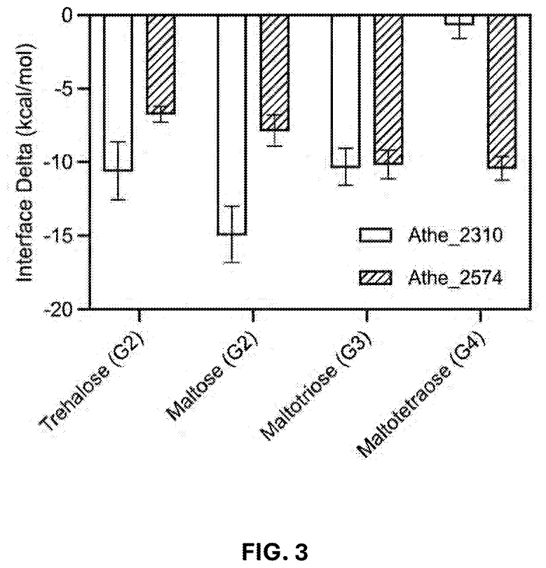

FIG. 3 illustrates interface energy deltas, corresponding to free energies of binding, for each simulated maltodextrin-binding protein and substrate combination. As shown in FIG. 3, the rank-ordering of each protein-substrate combination from computational modeling is aligned with experimental results from isothermal titration calorimetry and differential scanning calorimetry measurements. Athe_2310 exhibits more favorable binding energies with shorter maltodextrins such as trehalose and maltose, while Athe_2574 demonstrates progressively stronger binding with increasing maltodextrin chain length.

The distinct substrate specificities of Athe_2310 and Athe_2574 arise from differences in binding pocket architecture. Athe_2310 possesses two subsites available for ligand binding, which accommodates disaccharides. Athe_2574 possesses four subsites in the binding pocket, enabling accommodation of longer maltodextrin chains. The pocket volume of Athe_2574 is larger than that of Athe_2310, which correlates with the ability of Athe_2574 to bind longer oligosaccharides.

The presence of two ABC transporters for uptake of maltodextrins of different sizes in A. bescii enables efficient scavenging of alpha-glucan substrates. A bioenergetic benefit to uptaking higher-order maltodextrins may explain the affinity of Athe_2574 for longer oligosaccharides, as a fixed cost of two ATP molecules per translocation of one sugar molecule implies that longer sugars provide higher ATP rates of return. Possession of an ABC transporter for smaller substrates allows A. bescii to mitigate risk from the extracellular availability of both long and short oligosaccharides.

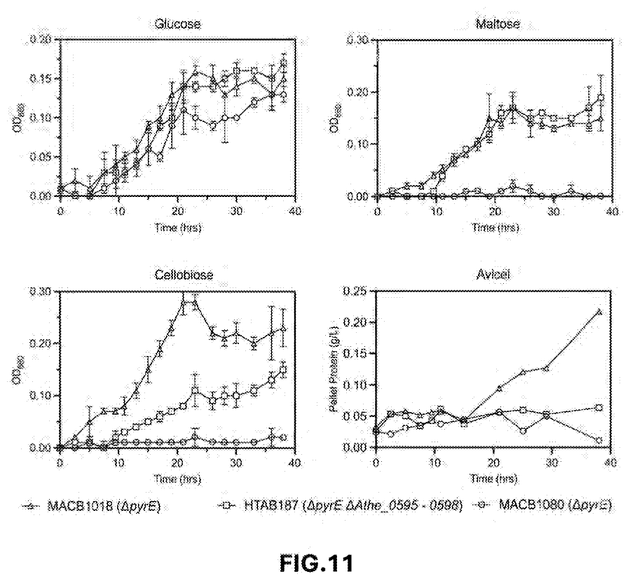

FIG. 11 illustrates growth curves of axenic, engineered A. bescii strains MACB1018, MACB1080, and HTAB187 on carbohydrate substrates glucose, maltose, cellobiose, and microcrystalline cellulose (Avicel). As shown in FIG. 11, growth curves may be performed in triplicate on individual carbon sources. For growth curves on glucose, maltose, and cellobiose, each data point may represent the mean Optical Density at 680 nm (OD₆₈₀) value, with error bars denoting the standard deviation. Growth curves on Avicel may be performed in triplicate, with time point samples mixed prior to pellet protein quantitation.

In some aspects, the genetically engineered bacterial strain is grown at a temperature of approximately 70 degrees Celsius for growth curve experiments. Cultures may be inoculated into modified nutrient source containing a single carbohydrate source at a density of 5 g/L and grown at a temperature of 70 degrees Celsius, without shaking, for 40 hours in biological triplicate. The OD₆₈₀ of growth cultures may be measured at intervals of approximately 2.5 to 6.0 hours. Cell protein content as a proxy for cell density under Avicel growth conditions may be quantified using the Bradford method with bovine serum albumin protein standards. A. bescii exhibits an optimal growth temperature of 75 to 78 degrees Celsius.

As shown in FIG. 11, all three strains demonstrate similar growth behavior on glucose. Strain HTAB187 behaves similarly to strain MACB1018 on glucose, while strain MACB1080 exhibits a slightly lower growth rate compared to strain MACB1018 during the exponential phase. Strain HTAB187 also exhibits no deviations in growth behavior compared to strain MACB1018 on maltose, while growth on maltose is impaired for strain MACB1080 due to inactivation of maltodextrin transporters through loss of the MsmK ATPase. On cellobiose, growth is impaired for strain HTAB187 compared to strain MACB1018, though the defect does not prevent growth entirely. Strain MACB1080 shows no growth on cellobiose. Strain HTAB187 is incapable of growth on cellulose, and strain MACB1080 also shows no growth on cellulose. Strain MACB1018 exhibits growth on cellulose. Deletion of the Athe_0595-0598 cello-oligosaccharide transporter locus results in impairment of growth on cellobiose and elimination of growth on cellulose, without apparent defects in the uptake of other glucose-based substrates such as glucose or maltose.

In some aspects, substrate-binding proteins are heterologously produced in E. coli BL21 (DE3) pRosetta2 for in vitro biophysical characterization. Genes encoding substrate-binding proteins absent their respective signal peptides may be amplified via PCR from A. bescii genomic DNA. N-terminal hexa-histidine-tags may be added to each substrate-binding protein gene to enable protein purification via immobilized metal affinity chromatography. E. coli BL21 (DE3) pRosetta2 strains transformed with respective expression vectors may be grown in auto-induction media at a temperature of 37 degrees Celsius and shake speed of 250 rpm.

In some aspects, biophysical characterization of substrate-binding proteins uses differential scanning calorimetry (DSC). DSC is useful for screening the carbohydrate specificity of extremely thermophilic substrate-binding proteins. DSC may reach temperatures as high as 130 degrees Celsius, enabling denaturation of A. bescii substrate-binding proteins that possess native melting temperatures above 92 degrees Celsius. In DSC, a constant rate of heating raises the heat capacity of the protein until the protein fully denatures. The temperature corresponding to the maximum heat capacity of the protein, implying complete denaturation, is the native melting temperature. The presence of cognate ligands may induce substrate-binding proteins to adopt a more thermally stable closed conformation, resulting in an increase in melting temperature. DSC measurements may be performed with a scan rate of 180 to 240 degrees Celsius per minute and a maximum temperature of 130 degrees Celsius. Each protein-sugar mixture may contain final concentrations of approximately 2 to 2.5 mg/mL protein and 5 mM sugar.

In some aspects, biophysical characterization of substrate-binding proteins uses isothermal titration calorimetry (ITC). ITC measurements may be performed at a temperature of 25 degrees Celsius. Standard titration experiments may be performed with a protein sample concentrated to 50 micromolar and a sugar ligand concentrated to 500 micromolar. The fitted isotherms yield the binding dissociation constant and other thermodynamic parameters. The slope of the binding isotherm at the equivalence point may be used to determine the dissociation constant.

A system for bioprocessing may comprise a first genetically engineered bacterial strain and a second genetically engineered bacterial strain. The first genetically engineered bacterial strain may comprise a first disruption or inactivation of a first sugar transporter gene locus. The first disruption or inactivation may reduce or eliminate the ability of the first strain to utilize a first set of one or more sugar substrates for growth. The second genetically engineered bacterial strain may comprise a second disruption or inactivation of a second sugar transporter gene locus different from the first sugar transporter gene locus. The second disruption or inactivation may reduce or eliminate the ability of the second strain to utilize a second set of one or more sugar substrates for growth. The second set of one or more sugar substrates may be different from the first set of one or more sugar substrates. This configuration enables co-cultures of strains that utilize orthogonal sugar substrates, where each strain may be specialized to consume particular sugars in a mixed feed and produce particular molecule outputs without competing for the same carbon sources.

In some aspects, the first genetically engineered bacterial strain and the second genetically engineered bacterial strain are each derived from Anaerocellum bescii. Using strains derived from the same parent organism provides compatible growth temperatures and enables the creation of a mix-and-match set of strains that may be grown together with predictable behavior. In some cases, both strains are derived from A. bescii MACB1018 as the parent strain.

In some aspects, the first disruption or inactivation reduces or eliminates the ability of the first strain to utilize D-xylose or D-glucose for growth. The first sugar transporter gene locus may comprise the monosaccharide transporter locus Athe_0105-0107, which encodes a substrate-binding protein Athe_0105 that binds D-xylose and D-glucose. As described above, disruption or inactivation of the Athe_0105-0107 locus results in impairment of growth on D-xylose and D-glucose. In some cases, the first genetically engineered bacterial strain may further comprise a disruption or inactivation of the msmK gene encoding the promiscuous ATPase, such that the combination of the Athe_0105-0107 disruption or inactivation with the msmK disruption or inactivation eliminates the ability of the first strain to utilize D-xylose and D-glucose for growth. For example, strain HTAB809 comprising deletions of both the Athe_0105-0107 locus and msmK does not grow on D-xylose and D-glucose.

In some aspects, the second disruption or inactivation reduces or eliminates the ability of the second strain to utilize L-arabinose or D-galactose for growth. The second sugar transporter gene locus may comprise the monosaccharide transporter locus Athe_2264-2268, which encodes a substrate-binding protein Athe_2264 that binds L-arabinose and D-galactose. As described above, disruption or inactivation of the Athe_2264-2268 locus results in impairment of growth on L-arabinose and D-galactose. In some cases, the second genetically engineered bacterial strain may further comprise a disruption or inactivation of the msmK gene encoding the promiscuous ATPase, such that the combination of the Athe_2264-2268 disruption or inactivation with the msmK disruption or inactivation eliminates the ability of the second strain to utilize L-arabinose and D-galactose for growth. For example, strain HTAB804 comprising deletions of both the Athe_2264-2268 locus and msmK does not grow on L-arabinose and D-galactose.

The pairing of a first strain that cannot utilize D-xylose or D-glucose with a second strain that cannot utilize L-arabinose or D-galactose enables co-cultures that feed on orthogonal sugars to avoid substrate competition. For example, strain HTAB804, which grows on D-xylose and D-glucose but does not grow on L-arabinose and D-galactose, may be co-cultured with strain HTAB809, which grows on L-arabinose and D-galactose but does not grow on D-xylose and D-glucose. The two strains may be co-cultured together on orthogonal pairs of sugars, such as a combination of xylose and arabinose, or glucose and galactose. Varying the ratios of these sugars may give rise to distinct co-culture compositions. In some cases, co-culture compositions may be verified by quantitative PCR of the undeleted monosaccharide transporter and high-performance liquid chromatography sampling of soluble sugars and sugar depletion throughout growth.

In some aspects, the first genetically engineered bacterial strain and the second genetically engineered bacterial strain may each be specialized to consume particular sugars in a mixed feed and produce particular molecule outputs from the sugars the strains can consume. Each strain may be engineered to produce different target products, such as enzymes, fuels, chemicals, metabolites, or proteins. The strain ratios and their outputs may be controlled by the sugar input. This division of labor approach reduces metabolic burden on each strain by allowing individual strains to specialize in synergistic, strain-specific functions.

In some aspects, the system for bioprocessing may further comprise a nutrient source containing at least two different sugar substrates. The first genetically engineered bacterial strain and the second genetically engineered bacterial strain may each selectively utilize a different sugar substrate from the nutrient source. As described above, strains with disruption or inactivation of different sugar transporter gene loci exhibit distinct sugar utilization profiles, enabling each strain to consume particular sugars in a mixed feed without competing for the same carbon sources. The nutrient source may contain combinations of monosaccharides such as D-xylose, D-glucose, L-arabinose, and D-galactose, or combinations of monosaccharides and oligosaccharides, depending on the intended bioprocessing application.

In some aspects, the nutrient source may include a lignocellulosic biomass substrate. Lignocellulosic biomass provides a diverse range of sugars including pentose and hexose sugars in various oligomeric forms. The lignocellulosic biomass substrates may include wheat arabinoxylan, beechwood xylan, arabinan, switchgrass, and microcrystalline cellulose (Avicel). Wheat arabinoxylan contains arabinose side chains attached to a xylan backbone, providing both arabinose and xylose sugars upon degradation. Beechwood xylan provides xylose and xylo-oligosaccharides upon degradation. Arabinan provides arabinose sugars upon degradation. Switchgrass is a lignocellulosic feedstock that is primarily composed of xylose and glucose. Microcrystalline cellulose (Avicel) provides glucose and cello-oligosaccharides upon degradation.

In some aspects, the first genetically engineered bacterial strain, the second genetically engineered bacterial strain, or both, are configured to degrade the lignocellulosic biomass substrate to release one or more sugars. Anaerocellum bescii possesses carbohydrate-active enzymes (CAZymes) that degrade cellulose and hemicellulose into oligosaccharides and monosaccharides. The Glucan-Degradation-Locus (GDL) encoded by Athe_1860-Athe_1867 produces the CAZymes that primarily drive cellulose deconstruction in A. bescii. The native secretome of A. bescii maximizes glucan conversion into cello-oligosaccharides. Strains derived from A. bescii may retain the ability to degrade lignocellulosic biomass substrates while exhibiting modified sugar uptake capabilities due to disruption or inactivation of sugar transporter gene loci.

In some aspects, co-cultures grown on lignocellulosic biomass substrates may exhibit dynamic growth patterns based on the sugar composition of the substrate and the sugar utilization profiles of the strains. For wheat arabinoxylan, a strain that grows on arabinose may grow first as the secretome debranches arabinose from arabinoxylans, while a strain that grows on xylose may increase in abundance as xylose becomes available from xylan backbone degradation. For switchgrass, a strain that grows on xylose and glucose may dominate due to the composition of switchgrass being primarily xylose and glucose. The relative abundance of each strain in a co-culture may be influenced by the sugar composition of the lignocellulosic biomass substrate and the timing of sugar release during degradation.

In some aspects, the first genetically engineered bacterial strain may be engineered to produce a first target product and the second genetically engineered bacterial strain may be engineered to produce a second target product different from the first target product. Target products may include ethanol, acetone, acetoin, and 2,3-butanediol. Anaerocellum bescii may be genetically engineered to produce such target products from lignocellulose at industrially-relevant yields. Each strain in a co-culture system may be specialized to consume particular sugars in a mixed feed and produce particular molecule outputs from the sugars the strain can consume.

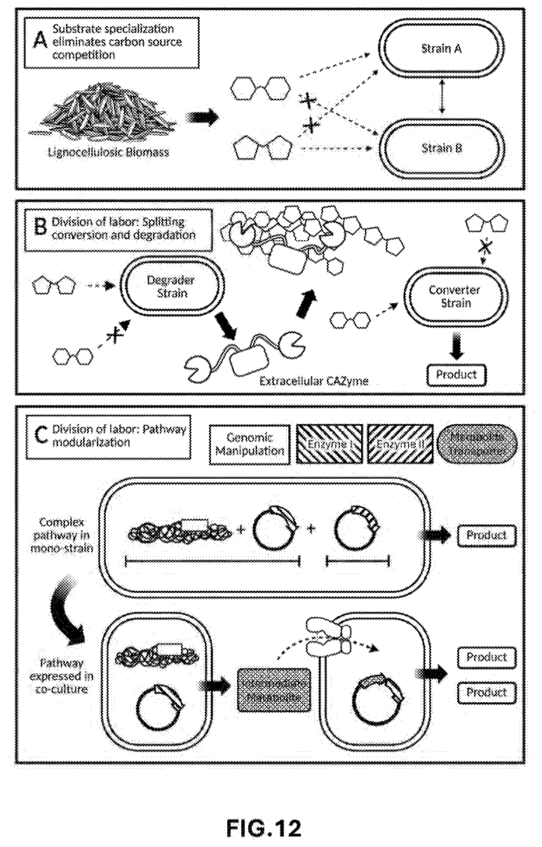

FIG. 12 illustrates biodesign strategies for synthetic thermophilic communities. As shown in FIG. 12, panel A depicts carbon substrate specialization as a strategy for engineered thermophilic communities. Microbes may be genetically engineered for removal of specialized substrate uptake pathways, eliminating competition with other thermophiles in co-culture. To avoid substrate competition, thermophilic microbes Strain A and Strain B may grow on distinct sugars from lignocellulosic biomass.

As further shown in FIG. 12, panel B depicts division of labor for consolidated bioprocessing of lignocellulosic biomass. In some aspects, the system may comprise strains where one strain is specialized for biomass deconstruction through expression of extracellular carbohydrate-active enzymes (CAZymes) while another strain is specialized for product synthesis with reduced CAZyme expression. A degrader strain may be specialized for deconstruction of lignocellulosic biomass, while a converter strain, which is minimally involved in biomass degradation, may instead focus on production of value-added chemicals. Splitting the roles of biomass degradation and conversion may decrease the metabolic burden placed on each strain, improving bioprocessing efficiency. Strains may be engineered to specialize in extracellular CAZyme expression without generating desired chemical products, or the production of a desired chemical coupled with reduced CAZyme expression.

As further shown in FIG. 12, panel C depicts pathway modularization across multiple strains. Complex biosynthetic pathways for bioproduction may be modularized across multiple strains. In the depicted example, a single strain may be overburdened by multiple genetic manipulations to generate a desired bioproduct. Burden sharing genetic modifications across two strains, while ensuring any pathway intermediate may be transported into the strain containing the final production step, may result in higher product titers. By distributing metabolic pathways more evenly across constituent strains, as opposed to expression of all pathway components within a single strain, the division of labor may offer a synergistic advantage by enhancing the fitness of each strain.

In some aspects, a ratio of the first target product to the second target product may be controlled by adjusting a ratio of sugar substrates provided to the first genetically engineered bacterial strain and the second genetically engineered bacterial strain. For example, if a first strain such as strain HTAB804 comprising deletions of both the Athe_2264-2268 locus and msmK is engineered to produce a first target product such as acetone, and a second strain such as strain HTAB809 comprising deletions of both the Athe_0105-0107 locus and msmK is engineered to produce a second target product such as ethanol, the initial composition of sugar inputs may dictate the composition of final product outputs. The ratio of sugar substrates, such as a ratio of D-xylose to L-arabinose, may control the ratio of target products, such as a ratio of acetone to ethanol produced. This approach enables control of co-culture compositions and product ratios through sugar inputs, providing a mechanism for flexible bioprocessing strategies.

In some aspects, the system for bioprocessing may be used as a platform technology with applications under different bioprocessing and manufacturing conditions. The engineered strains with tailored sugar utilization profiles may be deployed across a range of industrial settings, including batch fermentation, fed-batch fermentation, and continuous fermentation processes. The ability to control strain ratios and product outputs through sugar inputs provides flexibility for adapting the system to different feedstock compositions and product specifications. For aerobic or microaerobic bioprocessing conditions, the engineered strains may be cultured with controlled oxygen levels to optimize growth rates and product yields. For anaerobic bioprocessing conditions, the engineered strains may be cultured in the absence of oxygen to favor fermentative metabolism and production of reduced products such as ethanol. The system may be scaled from laboratory-scale bioreactors to pilot-scale and industrial-scale fermentation vessels, with the sugar-based control mechanism providing a consistent approach for managing co-culture dynamics across different scales. The platform technology may be adapted for different temperature regimes, including thermophilic conditions at 55 to 80 degrees Celsius for strains derived from Anaerocellum bescii, or mesophilic conditions for co-cultures that include mesophilic partner organisms. The system may also be integrated with downstream processing operations, where the selective sugar utilization by engineered strains simplifies product recovery by reducing the complexity of the fermentation broth. The platform technology may be applied to different lignocellulosic feedstocks, including agricultural residues such as corn stover and wheat straw, dedicated energy crops such as switchgrass and miscanthus, and forestry residues, with the co-culture composition adjusted based on the sugar profile of each feedstock.

A method of controlling microbial growth in a bioprocessing application may comprise providing at least one genetically engineered bacterial strain and growing the at least one genetically engineered bacterial strain in a nutrient source containing one or more sugar substrates.

In some aspects, the nutrient source may be selected based on the bioprocessing or manufacturing conditions. For laboratory-scale experiments, the nutrient source may comprise a defined minimal nutrient source containing individual sugar substrates at concentrations ranging from 1 to 10 g/L. For industrial-scale fermentation, the nutrient source may comprise complex media containing yeast extract, peptone, or corn steep liquor to provide nitrogen sources and growth factors. For consolidated bioprocessing applications, the nutrient source may comprise unpretreated or minimally pretreated lignocellulosic biomass substrates such as switchgrass, corn stover, or wheat straw. For fed-batch fermentation processes, the nutrient source may comprise an initial sugar concentration with additional sugar substrates added over time to maintain optimal growth conditions and prevent substrate inhibition. For continuous fermentation processes, the nutrient source may comprise a steady-state sugar concentration maintained through continuous feeding and withdrawal of culture broth. For high-temperature bioprocessing with thermophilic bacteria, the nutrient source may comprise heat-stable components that do not degrade or caramelize at temperatures of 70 to 80 degrees Celsius. For anaerobic fermentation, the nutrient source may be prepared under oxygen-free conditions with reducing agents such as cysteine or sodium sulfide to maintain a low redox potential. For co-culture applications, the nutrient source may comprise multiple sugar substrates at ratios designed to support the growth of each strain in the co-culture according to the desired population dynamics.

In some aspects, the nutrient source may be selected based on a desired environmental or downstream processing conditions. For processes where the fermentation broth will be subjected to distillation for product recovery, the nutrient source may comprise components that do not form azeotropes with the target products or that do not foul distillation equipment. For processes involving membrane separation or filtration, the nutrient source may comprise components that do not cause membrane fouling or that are compatible with the molecular weight cutoff of the separation membranes. For processes where the spent nutrient source will be recycled back to the fermentation vessel, the nutrient source may comprise components that remain stable through multiple fermentation cycles and do not accumulate inhibitory byproducts. For processes where the fermentation residue will be used as animal feed or fertilizer, the nutrient source may comprise components that are non-toxic and nutritionally beneficial for the intended downstream application. For processes conducted in outdoor or semi-controlled environments, the nutrient source may comprise components that are stable under variable temperature and humidity conditions. For processes where the target product is sensitive to pH changes, the nutrient source may comprise buffering agents that maintain pH within an optimal range throughout fermentation. For processes where product extraction will be performed using organic solvents, the nutrient source may comprise components that partition favorably into the aqueous phase and do not interfere with solvent extraction efficiency.

The at least one genetically engineered bacterial strain may comprise a genome having a disruption or inactivation of at least one sugar transporter gene locus. The disruption or inactivation may reduce or eliminate the ability of the strain to utilize at least one sugar substrate for growth while the strain retains the ability to utilize at least one other sugar substrate for growth. Growth of the at least one genetically engineered bacterial strain may be controlled based on the one or more sugar substrates present in the nutrient source.

In some aspects, by creating strains that cannot utilize particular sugars, the method may be used as a control strategy in monoculture or mixed cultures such that growth of the strains may be controlled by addition of the sugars the strains can grow on. The at least one genetically engineered bacterial strain may be grown on a sugar-containing substrate, including purified sugars or plant biomass, in individual or mixed culture. Depending on the strain or strain mixture chosen, all sugars may be utilized or some sugars may remain unconsumed.

In some aspects, the at least one genetically engineered bacterial strain is a thermophilic bacterium grown at a temperature of 55 degrees Celsius or higher. Thermophilic bacteria operate at elevated temperatures, providing process advantages including enhanced reaction kinetics, reduced risks of bioreactor contamination, and lower energy costs for temperature control. In some cases, the at least one genetically engineered bacterial strain may be grown at a temperature of approximately 70 degrees Celsius for growth experiments. In some cases, the at least one genetically engineered bacterial strain may be grown at a temperature of 75 to 78 degrees Celsius, corresponding to the optimal growth temperature range for Anaerocellum bescii.

In some aspects, growing may include culturing a plurality of genetically engineered bacterial strains in the nutrient source. Each strain of the plurality may have a different sugar transporter gene locus disruption or inactivation. A composition of the plurality of strains in the nutrient source may be controlled by adjusting a ratio of sugar substrates in the nutrient source. For example, if each strain utilizes a different sugar, different strains may be specialized for producing different molecules in a co-culture, and strain ratios and outputs may be controlled by the sugar input. Varying the ratios of sugars in the nutrient source may give rise to distinct co-culture compositions.

In some aspects, the nutrient source may comprise a lignocellulosic biomass substrate. The at least one genetically engineered bacterial strain may degrade the lignocellulosic biomass substrate to release one or more sugars. Lignocellulosic biomass provides a diverse range of sugars including pentose and hexose sugars in various oligomeric forms. The at least one genetically engineered bacterial strain may exhibit selective growth based on the ability of the strain to utilize particular sugars released from the lignocellulosic biomass.

In some aspects, the at least one genetically engineered bacterial strain may be derived from Anaerocellum bescii. The at least one sugar transporter gene locus may encode one or more components of an ATP-Binding Cassette transporter. As described above, A. bescii possesses multiple ABC sugar transporters with distinct sugar specificities, enabling the creation of strains with tailored sugar utilization profiles through targeted transporter disruption or inactivation.

In some aspects, the genome of the at least one genetically engineered bacterial strain may further comprise a disruption or inactivation of a gene encoding a promiscuous ATPase that powers a plurality of oligosaccharide ATP-Binding Cassette transporters. The disruption or inactivation of the gene encoding the promiscuous ATPase in combination with the disruption or inactivation of the at least one sugar transporter gene locus may eliminate the ability of the strain to utilize the at least one sugar substrate for growth. As described above, combining a monosaccharide transporter disruption or inactivation with disruption or inactivation of msmK may completely eliminate growth on selected monosaccharides, which may be advantageous when partial growth impairment from single transporter disruptions or inactivations is insufficient for the intended bioprocessing application.

In some aspects, the at least one genetically engineered bacterial strain may degrade the lignocellulosic biomass substrate while leaving unconsumed the at least one sugar substrate that the strain cannot utilize. The unconsumed sugar substrate may be available for utilization by a different organism in a subsequent process stage. Strains that cannot utilize particular sugars may grow on and degrade plant lignocellulosic biomass while leaving behind particular sugars that may then be fed to another organism in a subsequent process stage.

In some aspects, the different organism is a yeast. Yeast strains are well-established industrial organisms with robust fermentation capabilities. Pairing engineered thermophilic bacteria that leave behind specific sugars with yeast that efficiently convert those sugars enables hybrid bioprocessing approaches that leverage the strengths of each organism.

In some cases, the yeast may be Saccharomyces cerevisiae, which is widely used in industrial ethanol production and possesses well-characterized sugar metabolism pathways. In some cases, the yeast may be Pichia stipitis (also known as Scheffersomyces stipitis), which is capable of fermenting xylose and other pentose sugars that some S. cerevisiae strains cannot efficiently utilize. In some cases, the yeast may be Kluyveromyces marxianus, which exhibits thermotolerance at temperatures up to approximately 45 to 50 degrees Celsius and may be suitable for processes operating at moderately elevated temperatures. In some cases, the yeast may be an engineered strain of S. cerevisiae that has been modified to express xylose isomerase or xylose reductase and xylitol dehydrogenase pathways for improved pentose utilization.

Any appropriate microorganism may be used for co-culturing. In some cases, the different organism may be a filamentous fungus such as Trichoderma reesei, which produces cellulases and hemicellulases and may be paired with engineered bacterial strains in sequential or parallel bioprocessing configurations. In some cases, the different organism may be Aspergillus niger, which is capable of producing organic acids such as citric acid and gluconic acid from glucose and other hexose sugars. In some cases, the different organism may be Zymomonas mobilis, a bacterium with high ethanol productivity that efficiently ferments glucose and fructose but does not naturally utilize pentose sugars, making it suitable for pairing with engineered strains that leave behind hexose sugars. In some cases, the different organism may be Clostridium acetobutylicum or Clostridium beijerinckii, which are capable of producing acetone, butanol, and ethanol through solventogenic fermentation and may utilize sugars left unconsumed by the engineered thermophilic strains. In some cases, the different organism may be Corynebacterium glutamicum, which is widely used for amino acid production and may be engineered to utilize specific sugars in a sequential bioprocessing approach. In some cases, the different organism may be Lactobacillus species, which produce lactic acid from various sugars and may be deployed in processes where the engineered thermophilic bacteria leave behind specific monosaccharides. In some cases, the different organism may be Rhodosporidium toruloides or other oleaginous yeasts, which accumulate lipids from sugars and may convert unconsumed sugars into biodiesel precursors.

As described above, growing may include culturing a plurality of genetically engineered bacterial strains in a nutrient source. Each strain of the plurality may have a different sugar transporter gene locus disruption or inactivation. A composition of the plurality of strains in the nutrient source may be controlled by adjusting a ratio of sugar substrates in the nutrient source. For example, strains that utilize orthogonal sugar substrates may be co-cultured together, where varying the ratios of sugars in the nutrient source gives rise to distinct co-culture compositions.

In some aspects, the co-culture system may comprise strains that are pulsed with different ratios of orthogonal sugars over time to control population dynamics. Pulsing mixed sugars over time may be used to control co-culture growth dynamics. For example, co-culture growth dynamics may be measured in response to pulsing different ratios of D-xylose and L-arabinose over time. The timing and ratio of sugar pulses may influence the relative abundance of each strain in the co-culture, enabling dynamic control of population composition throughout a bioprocessing run.

In some aspects, the co-culture system may use conditioned media approaches where one strain is grown first to deplete specific sugars, and the filter-sterilized media is then used to grow a second strain on residual sugars. For example, a first strain such as strain HTAB804 may be grown on a nutrient source containing D-xylose and L-arabinose, where D-xylose is depleted over time while L-arabinose remains unconsumed. After filter-sterilizing the nutrient source, a second strain such as strain HTAB809 may be grown on the pre-conditioned, residual L-arabinose media. The conditioned media approach enables sequential cultivation of strains on orthogonal sugar substrates without direct competition for carbon sources.

In some aspects, co-culture composition may be quantified by quantitative polymerase chain reaction (qPCR) of the undeleted monosaccharide transporter and high-performance liquid chromatography (HPLC) sampling of soluble sugars and sugar depletion throughout growth. The qPCR method may target the monosaccharide transporter gene locus that remains intact in each strain, enabling differentiation between strains based on their distinct genotypes. HPLC sampling may be used to monitor the concentration of individual sugars in the nutrient source over time, providing data on sugar consumption rates and substrate depletion by each strain. The combination of qPCR and HPLC measurements enables verification of co-culture compositions and correlation of strain abundance with sugar utilization profiles.

In some aspects, the nutrient source may comprise a lignocellulosic biomass substrate. Lignocellulosic biomass provides a diverse range of sugars including pentose and hexose sugars in various oligomeric forms. The at least one genetically engineered bacterial strain may degrade the lignocellulosic biomass substrate to release one or more sugars. Lignocellulosic biomass substrates may include cellulose, xylans, arabinoxylans, and pectin. In some cases, the lignocellulosic biomass substrates may include wheat arabinoxylan, beechwood xylan, arabinan, switchgrass, and microcrystalline cellulose. Wheat arabinoxylan contains arabinose side chains attached to a xylan backbone, providing both arabinose and xylose sugars upon degradation. Beechwood xylan provides xylose and xylo-oligosaccharides upon degradation. Arabinan provides arabinose sugars upon degradation. Switchgrass is a lignocellulosic feedstock that is composed of xylose and glucose. Microcrystalline cellulose provides glucose and cello-oligosaccharides upon degradation.

In some aspects, the at least one genetically engineered bacterial strain is derived from Anaerocellum bescii. As described above, Anaerocellum bescii is an extremely thermophilic, lignocellulose-degrading bacterium that degrades the cellulose and hemicellulose components of lignocellulosic biomass into a diversity of oligosaccharide and monosaccharide sugars. A. bescii possesses carbohydrate-active enzymes (CAZymes) that degrade cellulose and hemicellulose into oligosaccharides and monosaccharides. The Glucan-Degradation-Locus (GDL) encoded by Athe_1860-Athe_1867 produces the CAZymes that drive cellulose deconstruction in A. bescii. The native secretome of A. bescii maximizes glucan conversion into cello-oligosaccharides. The secretome composition of A. bescii may adapt to external substrates, enabling efficient degradation of the diverse polysaccharides that make up lignocellulose. Strains derived from A. bescii may retain the ability to degrade lignocellulosic biomass substrates while exhibiting modified sugar uptake capabilities due to disruptions or inactivations of sugar transporter gene loci.

In some aspects, the at least one sugar transporter gene locus encodes one or more components of an ATP-Binding Cassette transporter. As described above, A. bescii possesses twenty-three ATP-Binding Cassette (ABC) sugar transporters that mediate uptake of diverse carbohydrates released from lignocellulose. ABC transporters comprise a hydrophobic transmembrane protein domain, a cytoplasmic ATPase domain, and an extracellular substrate-binding domain that helps determine the sugar specificity of the associated transporter. The at least one sugar transporter gene locus may encode at least one substrate-binding protein and at least one transmembrane domain protein. The substrate-binding protein binds and delivers sugars in the extracellular space to the transmembrane entryway, and the substrate-binding protein largely dictates an ABC transporter's substrate specificity.

In some aspects, the genome of the at least one genetically engineered bacterial strain further comprises a disruption or inactivation of a gene encoding a promiscuous ATPase that powers a plurality of oligosaccharide ATP-Binding Cassette transporters. The disruption or inactivation of the gene encoding the promiscuous ATPase in combination with the disruption or inactivation of the at least one sugar transporter gene locus may eliminate the ability of the strain to utilize the at least one sugar substrate for growth. In A. bescii, the promiscuous ATPase MsmK encoded by Athe_1803 powers twenty-one Carbohydrate-Uptake-Transporter Family 1 (CUT1) oligosaccharide transporters. Disruption or inactivation of msmK (Athe_1803) has been shown to eliminate growth on oligosaccharides such as cellobiose. However, disruption or inactivation of msmK (Athe_1803) does not prevent growth on monosaccharides, as the Carbohydrate-Uptake-Transporter Family 2 (CUT2) monosaccharide transporters possess their own respective ATPases. Among the twenty-one oligosaccharide transporters powered by MsmK, some may have slight affinity for monosaccharides. This affinity may explain why single transporter disruption or inactivation of Athe_0105-0107 or Athe_2264-2268 fail to completely eliminate growth on the monosaccharides that the deleted transporters have been determined to uptake. Combining a monosaccharide transporter disruption or inactivation with disruption or inactivation of msmK (Athe_1803) may completely eliminate growth on the selected monosaccharides. This combination of disruptions and/or inactivations provides a mechanism for achieving complete elimination of growth on targeted monosaccharides, which may be advantageous when partial growth impairment from single transporter disruptions or inactivations is insufficient for the intended bioprocessing application.

In some aspects, a genetically engineered bacterial strain may degrade a lignocellulosic biomass substrate while leaving unconsumed a sugar substrate that the strain cannot utilize. The unconsumed sugar substrate may be available for utilization by a different organism in a subsequent process stage. As described above, strains with disruption or inactivation of sugar transporter gene loci exhibit modified sugar uptake capabilities while retaining the ability to degrade lignocellulosic biomass through carbohydrate-active enzymes. Strains that cannot utilize particular sugars may grow on and degrade plant lignocellulosic biomass while leaving behind particular sugars that may then be fed to another organism in a subsequent process stage.

In some aspects, the different organism is a yeast. Yeast strains are well-established industrial organisms with robust fermentation capabilities and extensive genetic toolkits for metabolic engineering. Pairing engineered thermophilic bacteria that leave behind specific sugars with yeast that efficiently convert those sugars enables hybrid bioprocessing approaches that leverage the strengths of each organism. In such hybrid approaches, a thermophilic bacterium such as an engineered Anaerocellum bescii strain may perform biomass deconstruction at elevated temperatures, releasing a diverse range of sugars from lignocellulosic feedstocks. The engineered strain may consume certain sugars while leaving other sugars unconsumed due to disruptions or inactivations of the corresponding sugar transporter gene loci. The unconsumed sugars may then be provided to a yeast strain in a subsequent process stage, where the yeast may convert the sugars into target products such as ethanol or other fermentation products. This sequential approach enables each organism to operate under conditions suited to its physiology while collectively achieving conversion of lignocellulosic biomass into desired products.

In some aspects, the genetically engineered bacterial strain may be co-cultured with other thermophilic bacteria that possess complementary sugar utilization profiles. The co-culture system may include Clostridium thermocellum, which effectively uptakes cello-oligosaccharides but cannot utilize xylose or arabinose. Clostridium thermocellum may be paired with Thermoanaerobacter saccharolyticum, which can ferment pentose sugars released from hemicellulose. The pairing of C. thermocellum and T. saccharolyticum leverages native substrate specialization, where C. thermocellum consumes cello-oligosaccharides derived from cellulose degradation while T. saccharolyticum consumes pentose sugars such as xylose and arabinose derived from hemicellulose degradation. Consumption of pentose sugars by T. saccharolyticum may also prevent end-product inhibition of C. thermocellum hemicellulases, such that improved biomass hydrolysis may depend on the growth of both thermophiles.

In some aspects, the genetically engineered bacterial strain may be co-cultured with Thermoanaerobacterium aotearoense SCUT27 that has been modified to uptake and metabolize sucrose. As C. thermocellum cannot consume sucrose or pentose sugars but can consume hexose sugars, co-culturing C. thermocellum with T. aotearoense SCUT 27 may enable utilization of sugars from both lignocellulosic and sucrose-rich agricultural feedstocks. Engineered strains of A. bescii that cannot utilize starch or cellulose, relying on hemicellulose for growth, may likewise be co-cultured with C. thermocellum to achieve complementary substrate utilization.

In some aspects, the genetically engineered bacterial strain may be co-cultured with thermophilic Geobacillus strains such as Geobacillus thermoglucosidasius. Geobacillus thermoglucosidasius has been engineered to produce ethanol and lactic acid. Geobacillus strains may rely on dedicated lignocellulose-degrading strains to provide the hexose and pentose monosaccharides needed for growth. In such co-culture systems, an engineered A. bescii strain may degrade lignocellulosic biomass and release monosaccharides, while a Geobacillus strain may convert the released monosaccharides into target products such as ethanol or lactic acid. The choice of organisms in thermophilic synthetic communities may be driven by the ability to tailor the abilities of each organism, including biomass degradation, sugar transport, and metabolism, through genetic manipulation.

FIG. 22 illustrates growth curves demonstrating genetic elimination of sugar uptake in engineered A. bescii strains. As shown in FIG. 22, growth curves of wild-type strain MACB1018, strain HTAB804 carrying deletions of both the Athe_2264-2268 locus and msmK, strain HTAB809 carrying deletions of both the Athe_0105-0107 locus and msmK, and strain HTAB849 carrying deletions of the Athe_2264-2268 locus, the Athe_0105-0107 locus, and msmK are shown on D-xylose, D-glucose, L-arabinose, and D-galactose. The wild-type strain MACB1018 exhibits growth on all four monosaccharides. Strain HTAB804 grows on D-xylose and D-glucose but does not grow on L-arabinose and D-galactose, demonstrating that the combination of the Athe_2264-2268 deletion with the msmK deletion eliminates the ability of the strain to utilize L-arabinose and D-galactose for growth. Strain HTAB809 grows on L-arabinose but does not grow on D-xylose, D-glucose, or D-galactose, demonstrating that the combination of the Athe_0105-0107 deletion with the msmK deletion eliminates the ability of the strain to utilize D-xylose and D-glucose for growth. Strain HTAB849 does not grow on any of the four monosaccharides tested, demonstrating that the combination of both monosaccharide transporter deletions with the msmK deletion eliminates the ability of the strain to utilize D-xylose, D-glucose, L-arabinose, and D-galactose for growth.

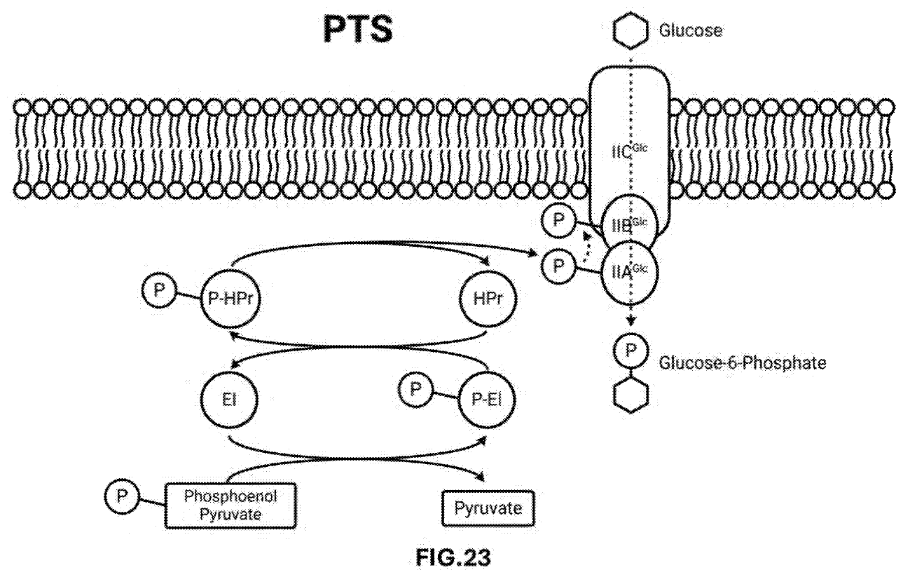

FIG. 23 illustrates a schematic of the Phosphotransferase System (PTS) for fructose transport in A. bescii. As shown in FIG. 23, the PTS comprises cytoplasmic phosphoryl transfer proteins including Enzyme I (EI), Histidine Protein (HPr), Enzyme IIA (EIIA), Enzyme IIB (EIIB), and an integral permease (IIC). Phosphoenolpyruvate serves as the phosphoryl donor, with phosphate groups transferred sequentially through EI, HPr, EIIA, and EIIB to the incoming fructose molecule as it traverses the membrane through IIC. The PTS transporter operates independently of the ABC transporter systems and provides an alternative mechanism for sugar uptake that couples transport with phosphorylation. The PTS fructose transporter locus is encoded by Athe_0148-Athe_0151 in A. bescii.

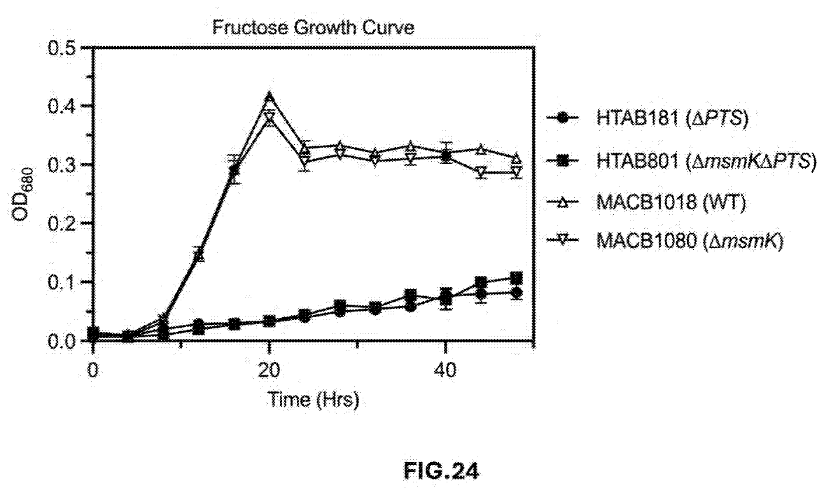

FIG. 24 illustrates growth curves confirming the PTS plays a role in fructose uptake. As shown in FIG. 24, growth curves of wild-type strain MACB1018, strain MACB1080 carrying the msmK deletion, strain HTAB181 carrying a deletion of the PTS fructose transporter locus (Athe_0148-Athe_0151), and strain HTAB801 carrying deletions of both msmK and the PTS fructose transporter locus are shown on fructose. Strains MACB1018 and MACB1080 with intact PTS transporters exhibit growth on fructose, while strains HTAB181 and HTAB801 carrying PTS deletions exhibit substantially reduced growth on fructose. The data demonstrates that deletion of the PTS locus disrupts growth on fructose, while deletion of msmK alone does not impair fructose utilization, consistent with fructose transport being mediated by the Phosphotransferase System transporter rather than an ATP-Binding Cassette transporter powered by the MsmK ATPase.

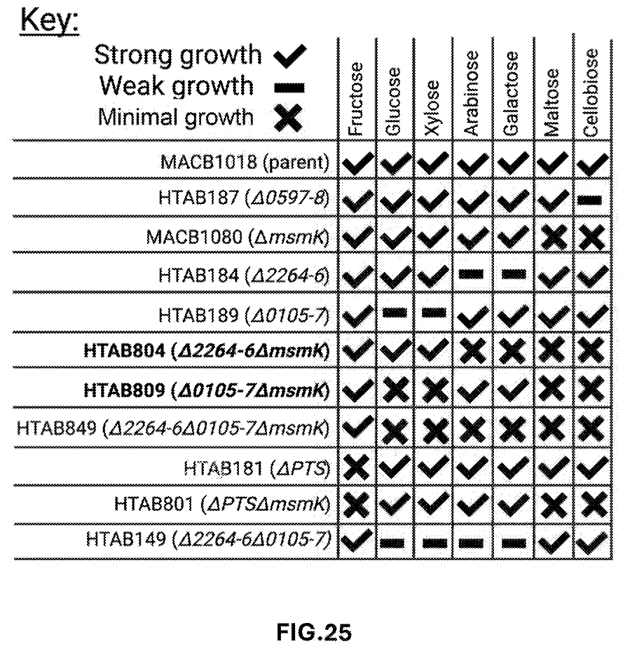

FIG. 25 illustrates a library of engineered A. bescii strains with their respective growth phenotypes on seven different sugar substrates. As shown in FIG. 25, the table summarizes growth capabilities of strains MACB1018, HTAB187, MACB1080, HTAB184, HTAB189, HTAB804, HTAB809, HTAB849, HTAB181, HTAB801, and HTAB149 on fructose, glucose, xylose, arabinose, galactose, maltose, and cellobiose. The table indicates strong growth, weak growth, or minimal growth for each strain-substrate combination. “Minimal” growth refers to situations with near elimination or elimination of growth as compared to the parent strain and/or wild type. “Weak growth” refers to situations with impaired growth, e.g., a reduction in growth as compared to the comparison parent strain. In some specific examples, this may involve situations where an Optical Density value for a strain, measured at a wavelength of 680 nm, is below of OD₆₈₀<a particular threshold (e.g., 0.10) at the end of a 40 hour growth curve at 70 °C “Strong” growth refers to situations with normal growth comparable to the parent strain and/or wile type. In some specific examples, this may refer to situations where the strain exhibits an OD₆₈₀>the particular threshold (again, e.g., 0.10) at the end of a 40 hour growth curve at 70 °C.

The library of strains demonstrates that combinatorial deletions of sugar transporter gene loci and the msmK gene produce strains with distinct sugar utilization profiles, enabling the creation of a mix-and-match set of strains that may be cultured together with predictable behavior based on the sugar substrates provided.

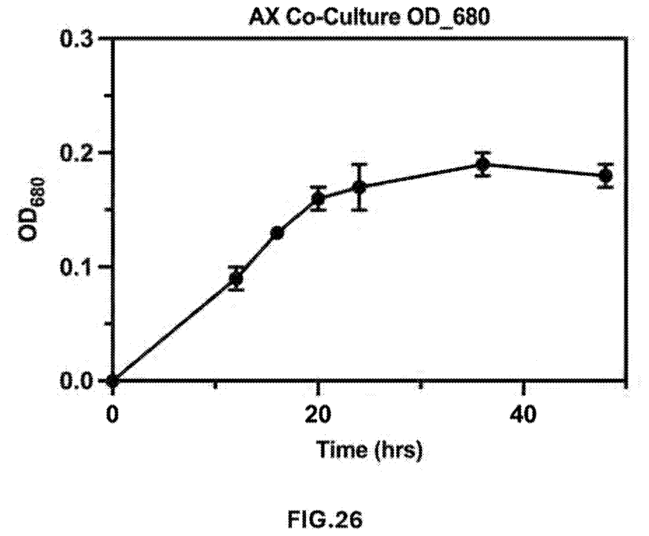

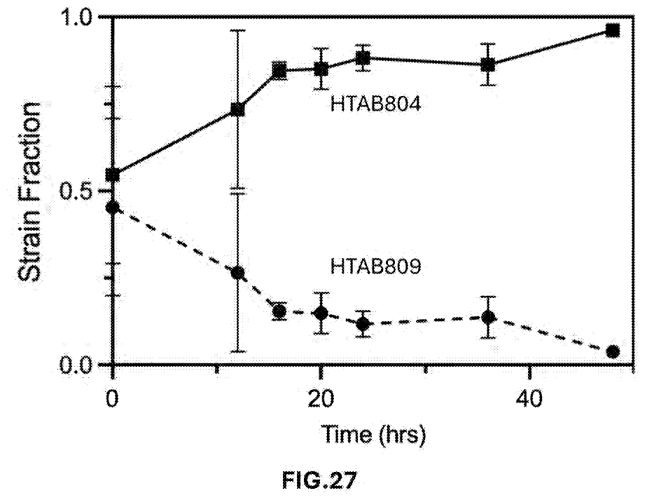

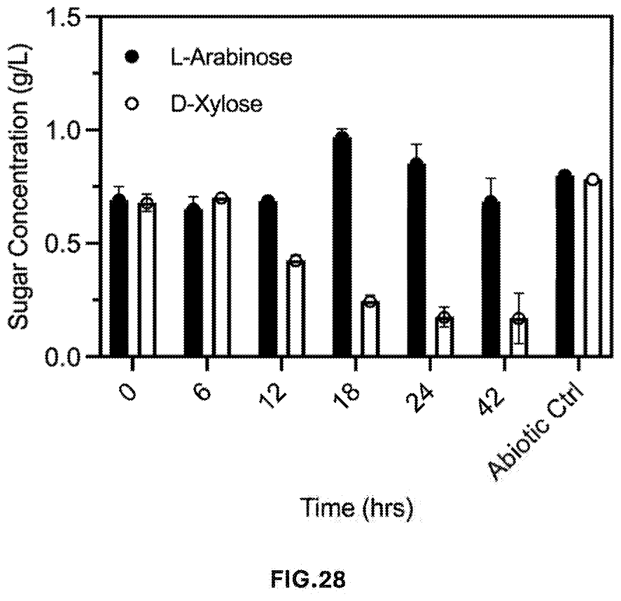

FIGS. 26, 27, and 28 illustrate co-culture experiments with engineered A. bescii strains HTAB804 and HTAB809 grown together on L-arabinose and D-xylose. As shown in FIG. 26, a co-culture growth curve demonstrates that a mixture of strains HTAB804 and HTAB809 results in a growing culture when inoculated at a 50/50 ratio. The co-culture exhibits increasing optical density over time, indicating that the two strains with orthogonal sugar utilization profiles may be cultured together on a medium containing both L-arabinose and D-xylose. As shown in FIG. 27, qPCR tracking of strain fraction over time demonstrates that the xylose-growing strain HTAB804 outcompetes the arabinose-growing strain HTAB809 in co-culture. The strain fraction of HTAB 804 increases from approximately 0.55 at time zero to approximately 0.95 by 50 hours, while the strain fraction of HTAB809 decreases correspondingly. As shown in FIG. 28, HPLC quantification of sugar content in the co-culture medium over time demonstrates gradual depletion of D-xylose while L-arabinose levels remain relatively stable. The D-xylose concentration decreases from approximately 0.7 grams per liter at time zero to approximately 0.2 grams per liter by 42 hours, while the L-arabinose concentration remains at approximately 0.7 to 0.95 grams per liter throughout the time course. The co-culture data demonstrates that strains with orthogonal sugar utilization profiles may be cultured together, with the relative abundance of each strain influenced by the sugar composition of the medium and the sugar consumption rates of each strain.

A number of implementations have been described. Nevertheless, it will be understood that various modifications may be made without departing from the spirit and scope of the disclosure. Accordingly, other implementations are within the scope of the following claims.

1. A genetically engineered bacterial strain, comprising:
a genome comprising a disruption or inactivation of at least one sugar transporter gene locus, wherein the disruption or inactivation reduces or eliminates an ability of the genetically engineered bacterial strain to utilize at least one sugar substrate for growth;
wherein the genetically engineered bacterial strain retains an ability to utilize at least one other sugar substrate for growth.

2. The genetically engineered bacterial strain of claim 1, wherein the genetically engineered bacterial strain is a thermophilic bacterium.

3. The genetically engineered bacterial strain of claim 2, wherein the genetically engineered bacterial strain is derived from Anaerocellum bescii.

4. The genetically engineered bacterial strain of claim 2, wherein the at least one sugar transporter gene locus comprises a cello-oligosaccharide transporter locus, and wherein the disruption or inactivation reduces or eliminates the ability of the genetically engineered bacterial strain to utilize cellobiose, cellodextrins, or cellulose for growth.

5. The genetically engineered bacterial strain of claim 2, wherein the at least one sugar transporter gene locus comprises a monosaccharide transporter locus, and wherein the disruption or inactivation reduces or eliminates the ability of the strain to utilize D-xylose or D-glucose for growth.

6. The genetically engineered bacterial strain of claim 2, wherein the at least one sugar transporter gene locus comprises a monosaccharide transporter locus, and wherein the disruption or inactivation reduces or eliminates the ability of the strain to utilize L-arabinose or D-galactose for growth.

7. The genetically engineered bacterial strain of claim 2, wherein the genome further comprises a disruption or inactivation of a gene encoding a promiscuous ATPase that powers a plurality of oligosaccharide ATP-Binding Cassette transporters, and wherein the disruption or inactivation of the gene encoding the promiscuous ATPase in combination with the disruption or inactivation of the at least one sugar transporter gene locus eliminates the ability of the genetically engineered bacterial strain to utilize the at least one sugar substrate for growth.

8. The genetically engineered bacterial strain of claim 7, wherein the gene encoding the promiscuous ATPase is msmK.

9. The genetically engineered bacterial strain of claim 1, wherein the at least one sugar transporter gene locus encodes one or more components of an ATP-Binding Cassette transporter.

10. The genetically engineered bacterial strain of claim 9, wherein the at least one sugar transporter gene locus encodes at least one substrate-binding protein and at least one transmembrane domain protein.

11. A system for bioprocessing, comprising:
a first genetically engineered bacterial strain comprising a first disruption or inactivation of a first sugar transporter gene locus, wherein the first disruption or inactivation reduces or eliminates an ability of the first genetically engineered bacterial strain to utilize a first set of one or more sugar substrates for growth; and
a second genetically engineered bacterial strain comprising a second disruption or inactivation of a second sugar transporter gene locus different from the first sugar transporter gene locus, wherein the second disruption or inactivation reduces or eliminates an ability of the second genetically engineered bacterial strain to utilize a second set of one or more sugar substrates for growth, the second set being different from the first set.

12. The system of claim 11, wherein the first genetically engineered bacterial strain and the second genetically engineered bacterial strain are each derived from Anaerocellum bescii.

13. The system of claim 12, wherein the first disruption or inactivation reduces or eliminates the ability of the first genetically engineered bacterial strain to utilize D-xylose or D-glucose for growth, and wherein the second disruption or inactivation reduces or eliminates the ability of the second genetically engineered bacterial strain to utilize L-arabinose or D-galactose for growth.

14. The system of claim 11, further comprising a nutrient source containing at least two different sugar substrates, wherein the first genetically engineered bacterial strain and the second genetically engineered bacterial strain each selectively utilizes a different sugar substrate from the nutrient source.

15. The system of claim 14, wherein the nutrient source comprises a lignocellulosic biomass substrate.

16. The system of claim 15, wherein the first genetically engineered bacterial strain, the second genetically engineered bacterial strain, or both, are configured to degrade the lignocellulosic biomass substrate to release one or more sugars.

17. The system of claim 11, wherein the first genetically engineered bacterial strain is engineered to produce a first target product and the second genetically engineered bacterial strain is engineered to produce a second target product different from the first target product.

18. The system of claim 17, wherein a ratio of the first target product to the second target product is controlled by adjusting a ratio of sugar substrates provided to the first genetically engineered bacterial strain and the second genetically engineered bacterial strain.

19. A method of controlling microbial growth in a bioprocessing application, comprising:
providing at least one genetically engineered bacterial strain comprising a disruption or inactivation of at least one sugar transporter gene locus, wherein the disruption or inactivation reduces or eliminates an ability of the strain to utilize at least one sugar substrate for growth while the at least one genetically engineered bacterial strain retains an ability to utilize at least one other sugar substrate for growth; and
growing the at least one genetically engineered bacterial strain in a nutrient source containing one or more sugar substrates, wherein growth of the at least one genetically engineered bacterial strain is controlled based on the one or more sugar substrates present in the nutrient source.

20. The method of claim 19, wherein the at least one genetically engineered bacterial strain is a thermophilic bacterium grown at a temperature of 55 degrees Celsius or higher.

21. The method of claim 19, wherein growing the at least one genetically engineered bacterial strain comprises growing a plurality of genetically engineered bacterial strains in the nutrient source, each strain of the plurality of genetically engineered bacterial strains having a different sugar transporter gene locus disruption or inactivation, and wherein a composition of the plurality of strains in the nutrient source is controlled by adjusting a ratio of sugar substrates in the nutrient source.

22. The method of claim 19, wherein the nutrient source comprises a lignocellulosic biomass substrate, and wherein the at least one genetically engineered bacterial strain degrades the lignocellulosic biomass substrate to release one or more sugars.

23. The method of claim 19, wherein the at least one genetically engineered bacterial strain is derived from Anaerocellum bescii, and wherein the at least one sugar transporter gene locus encodes one or more components of an ATP-Binding Cassette transporter.

24. The method of claim 23, wherein the genome of the at least one genetically engineered bacterial strain further comprises a disruption or inactivation of a gene encoding a promiscuous ATPase that powers a plurality of oligosaccharide ATP-Binding Cassette transporters, and wherein the disruption or inactivation of the gene encoding the promiscuous ATPase in combination with the disruption or inactivation of the at least one sugar transporter gene locus eliminates the ability of the at least one genetically engineered bacterial strain to utilize the at least one sugar substrate for growth.

25. The method of claim 22, wherein the at least one genetically engineered bacterial strain degrades the lignocellulosic biomass substrate while leaving unconsumed the at least one sugar substrate that the at least one genetically engineered bacterial strain cannot utilize, and wherein the unconsumed sugar substrate is available for utilization by a different organism in a subsequent process stage.

26. The method of claim 25, wherein the different organism is a yeast.
